(19) United States
(12) Patent Application Publication
(10) Pub. No.: US 2003/0036504 A1
(43) Pub. Date: Feb. 20, 2003

(54) Use of exendins and agonists thereof for modulation of triglyceride levels and treatment of dyslipidemia

(75) Inventors: Orville G. Kolterman, Poway, CA (US); Andrew A. Young, Point Loma, CA (US)

Correspondence Address: BROBECK, PHLEGER & HARRISON LLP, 12390 EL CAMINO REAL, SAN DIEGO, CA, 92130, US

(73) Assignee: Amylin Pharmaceuticals, Inc.

(21) Appl. No.: 09/756,690

(22) Filed: Jan. 9, 2001

Related U.S. Application Data
(60) Provisional application No. 60/175,365, filed on Jan. 10, 2000.

(51) Int. Cl. A61K 38/17

(52) U.S. Cl. 514/12

(57) ABSTRACT

Methods for modulating the levels of plasma triglyceride and other lipids in a subject which comprise administration of an effective amount of an exendin or an exendin agonist, alone or in conjunction with other compounds or compositions that lower blood triglyceride and/or other lipid levels.

### FIELD OF THE INVENTION

The present invention relates to methods for modulating triglyceride levels comprising administration of an effective amount of an exendin or an exendin agonist, alone or in conjunction with other compounds or compositions that may affect triglyceride levels. Pharmaceutical compositions for use in the methods of the invention are also disclosed.

### BACKGROUND

The following description summarizes information that may be relevant to the present invention. It is not an admission that any of the information provided herein is prior art to the presently claimed inventions, or relevant, nor that any of the publications specifically or implicitly referenced are prior art.

### Triglycerides and Triglyceride Levels

Triglycerides are a type of fat called lipids, and they are the chemical form in which most fat exists in food as well as in the body. More than 90 percent of the fat in the food people eat and in the fat stores in their bodies is made up of triglycerides. The liver also makes triglycerides from alcohol or excess carbohydrates. Calories ingested in a meal that are not used immediately by tissues are converted to triglycerides and transported to fat cells to be stored. When triglycerides reach fat cells, an enzyme called lipoprotein lipase separates them from carrier molecules so they can be stored as fat. Hormones regulate the release of triglycerides from fat tissue to meet the needs of the body for energy between meals. The other two main classes of fats are phospholipids, such as lecithin, and sterols, such as cholesterol.

Like cholesterol, triglycerides are a necessary component of the chemistry of the body. Triglycerides circulate constantly in the blood, ferrying the fat-soluble vitamins A, D, E and K to locations where they are needed, aiding in the synthesis of certain hormones, and protecting cell membranes. Unlike cholesterol, triglyceride particles are large and do not enter the blood vessels and contribute to arterial blockages in the same way cholesterol does. High triglyceride levels, however, do indicate a defect in the system and have recently been confirmed as an early warning of heart trouble.

An excess amount of triglycerides in plasma is called hypertriglyceridemia. Hypertriglyceridemia is linked to the occurrence of coronary artery disease in some people. Elevated triglycerides may be a consequence of other disease, such as diabetes mellitus. E.g., “Management of Dyslipidemia in Adults With Diabetes,” Diabetes Care 22:556-559 (January 1999). Like cholesterol, increases in triglyceride levels can be detected by plasma measurements. Triglyceride levels vary from day to day and in response to meals, and these measurements should be made after an overnight food and alcohol fast. At least two separate tests may be required to get an accurate reading. The triglyceride level in a patient is indicative of various potential disorders.

Traditionally, for example, a triglyceride level below 200 mg/dl was considered normal. However, recent research suggests that to prevent heart disease, the optimal level of triglycerides is less than 150 mg/dl and, more preferably, less than 100 mg/dl. Researchers reported that in one study, conducted at the University of Maryland Medical Center in Baltimore, patients with triglyceride levels above 100 mg/dL had an increased risk of suffering from coronary events (more than twofold). Another study, conducted at the Rush Medical College in Chicago, reported that triglyceride levels above 190 made the blood considerably more viscous. Other studies have reportedly shown a correlation between blood viscosity and heart disease.

Accordingly, triglyceride levels between 200-700 mg/dl are believed to represent an increased risk of heart disease. At these levels, lipoprotein lipase enzyme is present, but it does not work well. Triglycerides increase in the blood and become part of the plaque that clogs arteries. Often people with high triglycerides also have low levels of the protective HDL cholesterol, further increasing the risk of heart disease. This pattern is also frequently found in diabetes.

Triglyceride levels of 1000 mg/dl or more represent an increased risk of pancreatitis. In this situation, lipoprotein lipase is absent and triglycerides can cause inflammation of the pancreas (pancreatitis). Heart disease risk is less of a concern because the triglyceride particles remain attached to the carrier molecules, which are too big to become part of the artery-clogging plaque.

In sum, based on measurements of fasting plasma triglyceride levels, triglyceride levels have been characterized as follows:

| | | |
|---|---|---|
| Normal triglycerides | Less than 100-200 | mg/dL |
| Borderline-high triglycerides | 200-400 | mg/dL |
| High triglycerides | 400-1000 | mg/dL |
| Very high triglycerides | Greater than 1000 | mg/dL |

Elevated triglycerides can be caused by diet (fatty foods, sweets, fruit juices, and alcohol can all increase levels), as well as by genetic factors. Thus, changes in life habits are a main therapy for higher than normal fasting triglycerides. The changes include cutting down on calorie intake, reducing saturated fat and cholesterol content of the diet, reducing alcohol intake, and committing to a regular exercise program. Because other risk factors for coronary artery disease multiply the hazard from hyperlipidemia, hypertension and cigarette smoking are also to be controlled. Even if drugs are used for treatment of hypertriglyceridemia, dietary management is still important.

It has been reported that elevated postprandial triglyceride levels are associated with cardiovascular disease. E.g., Karpe, J. Internal Med. 246:341-355 (1999), Karpe et al., Metabolism 48:301-307 (1999), Karpe et al., Atherosclerosis 141:307-314 (1998), Nikkila et al., Atherosclerosis 106:149-157 (1994), and Patsch et al., Atherosclerosis and Thrombosis 12:136-1345 (1992).

### Current Clinical Therapy for Elevated Triglycerides

As noted, many people strive to reduce triglyceride levels through exercise and a low-fat, low-sugar diet. The current therapeutic approach for elevated triglyceride levels is to control plasma triglycerides with medication. A large number of people with coronary heart disease have elevated triglyceride readings. Thus, doctors often recommend that such patients take drugs in addition to altering their diets to lower these counts. There are several triglyceride-lowering drugs currently available. The following table lists some of the major therapies used for the treatment of hyperlipidemia, including elevated triglycerides.

| Drug or Drug | Major | | |
|---|---|---|---|
| Type | Indications | Mechanism | Common Side Effects |
| Bile acid | Elevated | Promote bile acid excretion and | Bloating, constipation, elevated |
| sequestrants | LDL | increase LDL receptors in liver | triglycerides |
| cholestyramine | | | |
| colestipol | | | |
| Nicotinic acid | Elevated | Decreases VLDL synthesis | Cutaneous flushing, GI upset, |
| | LDL, VLDL | | elevated glucose, uric acid, and |
| | | | liver function tests |
| HMG CoA | Elevated | Inhibit cholesterol synthesis and | Myositis (muscle |
| reductase | LDL | upregulate LDL receptors in liver | inflammation), arthralgias (joint |
| inhibitors | | | pains), GI upset, elevated liver |
| (“statins”) | | | function tests |
| pravastatin | | | |
| simvastatin | | | |
| atorvastatin | | | |
| fluvastatin | | | |
| lovastatin | | | |
| Fibric acid | Elevated | Stimulate lipoprotein lipase (an | Myositis (muscle |
| derivatives | triglycerides, | enzyme that breaks down lipids in | inflammation), GI upset, |
| gemfibrozil | elevated | lipoproteins), may decrease | gallstones, elevated liver |
| | remnants | VLDL synthesis | function tests |
| Fish oils | Elevated | Decrease synthesis and increase | Diarrhea, GI upset, fishy odor |
| | triglycerides | breakdown of triglycerides | breath |

Thus, it can be appreciated that an effective means to control triglyceride and other lipid levels is an important and a major challenge. A superior method of treatment would be of great utility. Methods for controlling triglyceride and other lipid levels, and compounds and compositions which are useful therefor, have been invented and are described and claimed herein.

### Exendins and Exendin Agonists

Exendins are peptides that were first isolated form the salivary secretions of the Gila monster, a lizard found in Ariz., and the Mexican Beaded Lizard. Exendin-3 is present in the salivary secretions of Heloderma horridum, and exendin-4 is present in the salivary secretions of Heloderma suspectum (Eng, J., et al., J. Biol. Chem., 265:20259-62, 1990; Eng., J., et al., J. Biol. Chem., 267:7402-05, 1992). The exendins have some sequence similarity to several members of the glucagon-like peptide family, with the highest homology, 53%, being to GLP-1[7-36]NH₂ (Goke, et al., J. Biol. Chem., 268:19650-55, 1993). GLP-1[7-36]NH₂, also known as proglucagon[78-107] and most commonly as “GLP-1.” GLP- 1 has an insulinotropic effect, stimulating insulin secretion from pancreatic β-cells. GLP-1 also inhibits glucagon secretion from pancreatic α-cells (Orskov, et al., Diabetes, 42:658-61, 1993; D'Alessio, et al., J. Clin. Invest., 97:133-38, 1996). GLP-1 is reported to inhibit gastric emptying (Williams B, et al., J Clin Endocrinol Metab 81 (1): 327-32, 1996; Wettergren A, et al., Dig Dis Sci 38 (4): 665-73, 1993), and gastric acid secretion. (Schjoldager BT, et al., Dig Dis Sci 34 (5): 703-8, 1989; O'Halloran DJ, et al., J Endocrinol 126 (1): 169-73, 1990; Wettergren A, et al., Dig Dis Sci 38 (4): 665-73, 1993). GLP-1[7-37], which has an additional glycine residue at its carboxy terminus, also stimulates insulin secretion in humans (Orskov, et al., Diabetes, 42:658-61, 1993). A transmembrane G-protein adenylate-cyclase-coupled receptor believed to be responsible for the insulinotropic effect of GLP-1 is reported to have been cloned from a β-cell line (Thorens, Proc. Natl. Acad. Sci. USA 89:8641-45 (1992)).

Exendin-4 potently binds at GLP-1 receptors on insulin-secreting βTC1 cells, at dispersed acinar cells from guinea pig pancreas, and at parietal cells from stomach; the peptide is also said to stimulate somatostatin release and inhibit gastrin release in isolated stomachs (Goke, et al., J. Biol. Chem. 268:19650-55, 1993; Schepp, et al., Eur. J. Pharmacol., 69:183-91, 1994; Eissele, et al., Life Sci., 55:629-34, 1994). Exendin-3 and exendin-4 were reported to stimulate cAMP production in, and amylase release from, pancreatic acinar cells (Malhotra, R., et al., Regulatory Peptides,41:149-56, 1992; Raufman, et al., J. Biol. Chem. 267:21432-37, 1992; Singh, et al., Regul. Pept. 53:47-59, 1994). The use of exendin-3 and exendin-4 as insulinotrophic agents for the treatment of diabetes mellitus and the prevention of hyperglycemia has been proposed (Eng, U.S. Pat. No. 5,424,286).

C-terminally truncated exendin peptides such as exendin-4[9-39], a carboxyamidated molecule, and fragments 3-39 through 9-39 have been reported to be potent and selective antagonists of GLP-1 (Goke, et al., J. Biol. Chem., 268:19650-55, 1993; Raufman, J. P., et al., J. Biol. Chem. 266:2897-902, 1991; Schepp, W., et al., Eur. J. Pharm. 269:183-91, 1994; Montrose-Rafizadeh, et al., Diabetes, 45(Suppl. 2):152A, 1996). Exendin-4[9-39] is said to block endogenous GLP-1 in vivo, resulting in reduced insulin secretion. Wang, et al., J. Clin. Invest., 95:417-21, 1995; D'Alessio, et al., J. Clin. Invest., 97:133-38, 1996). The receptor apparently responsible for the insulinotropic effect of GLP-1 has reportedly been cloned from rat pancreatic islet cell (Thorens, B., Proc. Natl. Acad. Sci. USA 89:8641-8645, 1992). Exendins and exendin-4[9-39] are said to bind to the cloned GLP-1 receptor (rat pancreatic β-cell GLP-1 receptor (Fehmann H C, et al., Peptides 15 (3): 453-6, 1994) and human GLP-1 receptor (Thorens B, et al., Diabetes 42 (11): 1678-82, 1993)). In cells transfected with the cloned GLP-1 receptor, exendin-4 is reportedly an agonist, i.e., it increases cAMP, while exendin[9-39] is identified as an antagonist, i.e., it blocks the stimulatory actions of exendin-4 and GLP-1. Id.

Exendin-4[9-39] is also reported to act as an antagonist of the full length exendins, inhibiting stimulation of pancreatic acinar cells by exendin-3 and exendin-4 (Raufman, et al., J. Biol. Chem. 266:2897-902, 1991; Raufman, et al., J. Biol. Chem., 266:21432-37, 1992). It is also reported that exendin[9-39] inhibits the stimulation of plasma insulin levels by exendin-4, and inhibits the somatostatin release-stimulating and gastrin release-inhibiting activities of exendin-4 and GLP-1 (Kolligs, F., et al., Diabetes, 44:16-19, 1995; Eissele, et al., Life Sciences, 55:629-34, 1994).

Methods for regulating gastrointestinal motility using exendin agonists are described and claimed in U.S. application Ser. No. 08/908,867, filed Aug. 8, 1997, entitled, “Methods for Regulating Gastrointestinal Motility,” which application is a continuation-in-part of U.S. application Ser. No. 08/694,954, filed Aug. 8, 1996, which enjoys common ownership with the present invention and is hereby incorporated by reference.

Methods of reducing food intake using exendin agonists are described and claimed in U.S. application Ser. No. 09/003,869, filed Jan. 7, 1998, entitled, “Use of Exendin and Agonists Thereof for the Reduction of Food Intake,” claiming the benefit of Provisional Application Nos. 60/034,905, filed Jan. 7, 1997, 60/055,404, filed Aug. 7, 1997, 60/065,442 filed Nov. 14, 1997, and 60/066,029 filed Nov. 14, 1997. These applications also enjoy common ownership with the present invention and are hereby incorporated by reference.

Exendins have also been reported to have inotropic and diuretic effects. International Application No. PCT/US99/02554, filed Feb. 5, 1999, 1998, claiming the benefit of Provisional Application No. 60/075,122, filed Feb. 13, 1998. These applications also enjoy common ownership with the present invention and are hereby incorporated by reference.

Additionally, exendins have been reported to suppress glucagon secretion (United States Provisional Application No.60/132,017, entitled, “Methods for Glucagon Suppression,” filed Apr. 30, 1999, which enjoys common ownership with the present invention and is hereby incorporated by reference).

Exendin [9-39] has been used to investigate the physiological relevance of central GLP-1 in control of food intake (Turton, M. D. et al. Nature 379:69-72, 1996). GLP-1 administered by intracerebroventricular injection inhibits food intake in rats. This satiety-inducing effect of GLP-1 delivered ICV is reported to be inhibited by ICV injection of exendin [9-39] (Turton, supra). However, it has been reported that GLP-1 does not inhibit food intake in mice when administered by peripheral injection (Turton, M. D., Nature 379:69-72, 1996; Bhavsar, S. P., Soc. Neurosci. Abstr. 21:460 (188.8), 1995).

### SUMMARY OF THE INVENTION

The present invention concerns the discovery that exendins and exendin agonists have a significant effect on the reduction of blood serum triglyceride concentrations, rendering them ideal agents for the treatment of elevated triglycerides, which are associated with increased coronary heart disease.

The present invention is directed to novel methods for modulating triglyceride levels, as well as novel methods for the treatment of subjects with dyslipidemia (i.e., increased LDL cholesterol, increased VLDL cholesterol, and/or decreased HDL cholesterol), comprising the administration of an exendin, for example, exendin-3 [SEQ ID NO. 1: His Ser Asp Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala Pro Pro Pro Ser-NH₂], or exendin-4 [SEQ ID NO. 2: His Gly Glu Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala Pro Pro Pro Ser-NH₂], or other compounds which effectively bind to a receptor at which exendin exerts its actions which are beneficial in the treatment of undesired triglyceride levels.

In a first aspect, the invention features a method of modulating triglyceride levels in a subject comprising administering to the subject a therapeutically effective amount of an exendin or an exendin agonist. By an “exendin agonist” is meant a compound that mimics the effects of exendin in the modulation of triglyceride levels, for example, by binding to the receptor or receptors where exendin causes one or more of these effects, or by activating the signalling cascade by which exendin causes one or more of these effects.

Exendin agonist compounds include exendin acids, for example exendin-3 acid [SEQ ID NO. 185: His Ser Asp Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala Pro Pro Pro Ser] and exendin-4 acid [SEQ ID NO. 186: His Gly Glu Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala Pro Pro Pro Ser]. Preferred exendin agonist compounds include those described in International Application No. PCT/US98/16387, entitled, “Novel Exendin Agonist Compounds,” filed Aug. 6, 1998, claiming the benefit of United States Provisional Patent Application Serial No. 60/055,404, filed Aug. 8, 1997; International Application No. PCT/US98/24220 entitled, “Novel Exendin Agonist Compounds,” filed Nov. 13, 1998, claiming priority on United States Provisional Patent Application Serial No. 60/065,442, filed Nov. 14, 1997; and International Application No. PCT/US98/24273 entitled, “Novel Exendin Agonist Compounds,” filed Nov. 13, 1998, claiming priority on United States United States Provisional Patent Application Serial No. 60/066,029, filed Nov. 14, 1997; all of which enjoy common ownership with the present application and all of which are incorporated by this reference into the present application as though fully set forth herein. Additional preferred exendin agonist compounds are those described and claimed in United States Provisional Application Serial No. 60/132,018, entitled, “Modified Exendins and Exendin Agonists,” filed Apr. 30, 1999, which enjoys common ownership with the present application and which is incorporated by this reference into the present application as though fully set forth herein. Preferred exendin agonists are exendin analogs and derivatives. By exendin analog or derivative is meant a variant of the exendin molecule. The variant may be a naturally occurring allelic variant of an exendin or a non-naturally occurring variant of an exendin, such as those identified herein. Variants include deletion variants, substitution variants, and addition or insertion variants. Exendin analogs or derivatives will normally have an activity about 1% to about 10,000% of the activity of the exendin of which it is an analog or derivative. Other exendin analogs or derivatives will preferably have an activity about 10% to about 1,000% of the activity of the exendin of which it is an analog or derivative, more preferably an activity about 50% to about 500% of the activity of the exendin of which it is an analog or derivative. Most preferred exendin analogs or derivatives will have at least about 50% sequence similarity to the exendin of which it is an analog or derivative. Still more preferred exendig analogs or derivatives will have at least about 70%, or at least about 90%, or 95% sequence similarity to the exendin of which it is an analog or derivative.

By “elevated triglyceride levels” or “ETL” is meant any degree of triglyceride levels that is determined to be undesireable or is targeted for modulation.

Thus, in a first embodiment, the present invention provides a method for modulating triglyceride levels in a subject comprising administering to said subject a therapeutically effective amount of an exendin or an exendin agonist.

In one aspect, the modulation of triglyceride levels in a subject is modululation of fasting triglyceride levels. In another aspect, the modulation of triglyceride levels in a subject is modulation of postprandial (post-meal) triglyceride levels. In still another aspect, the modulation of triglyceride levels in a subject is the modululation of both fasting and postprandial triglyceride levels.

In yet another aspect, the modulation of lipid levels in a subject is modululation of fasting lipid levels. In another aspect, the modulation of lipid levels in a subject is modulation of postprandial (post-meal) triglyceride levels. In a further aspect, the modulation of lipid levels in a subject is the modululation of both fasting and postprandial lipid levels. In this aspect, lipids refer to lipids in addition to triglycercides, including, for example, cholesterols.

Preferred exendin agonist compounds include those described in International Application Nos. PCT/US98/16387, PCT/US98/24220, and PCT/US98/24273, which have been incorporated by reference in the present application. Preferably, the subject is a vertebrate, more preferably a mammal, and most preferably a human. In preferred aspects, the exendin or exendin agonist is administered parenterally, more preferably by injection, for example, by peripheral injection. Preferably, about 1 μg-30 μg to about 1 mg of the exendin or exendin agonist is administered per day. More preferably, about 1-30 μg to about 500 μg, or about 1-30 μg to about 50 μg of the exendin or exendin agonist is administered per day. Most preferably, depending upon the weight of the subject and the potency of the compound administered, about 3 μg to about 50 μg of the exendin or exendin agonist is administered per day. Preferred doses based upon patient weight for compounds having approximately the potency of exendin-4 range from about 0.005 μg/kg per dose to about 0.2 μg/kg per dose. More preferably, doses based upon patient weight for compounds having approximately the potency of exendin-4 range from about 0.02 μg/kg per dose to about 0.1 μg/kg per dose. Most preferrably, doses based upon patient weight for compounds having approximately the potency of exendin-4 range from about 0.05 μg/kg per dose to about 0.1 μg/kg per dose. These doses are administered from 1 to 4 times per day, preferably from 1 to 2 times per day. Doses of exendins or exendin agonists will normally be lower if given by continuous infusion. Doses of exendins or exendin agonists will normally be higher if given by non-injection methods, such as oral, buccal, sublingual, nasal, pulmonary or skin patch delivery.

In one preferred aspect, the exendin or exendin agonist used in the methods of the present invention is exendin-3. In another preferred aspect, said exendin is exendin-4. Other preferred exendin agonists include exendin-4 (1-30) [SEQ ID NO 6: His Gly Glu Gly Thr Phe Thr Ser Asp Leu Ser Lys Gln Met Glu Glu Glu Ala Val Arg Leu Phe Ile Glu Trp Leu Lys Asn Gly Gly], exendin-4 (1-30) amide [SEQ ID NO 7: His Gly Glu Gly Thr Phe Thr Ser Asp Leu Ser Lys Gln Met Glu Glu Glu Ala Val Arg Leu Phe Ile Glu Trp Leu Lys Asn Gly Gly-NH₂], exendin-4 (1-28) amide [SEQ ID NO 40: His Gly Glu Gly Thr Phe Thr Ser Asp Leu Ser Lys Gln Met Glu Glu Glu Ala Val Arg Leu Phe Ile Glu Trp Leu Lys Asn-NH₂], ¹⁴Leu,²⁵Phe exendin-4 [SEQ ID NO 9: His Gly Glu Gly Thr Phe Thr Ser Asp Leu Ser Lys Gln Leu Glu Glu Glu Ala Val Arg Leu Phe Ile Glu Phe Leu Lys Asn Gly Gly Pro Ser Ser Gly Ala Pro Pro Pro Ser-NH₂], ¹⁴Leu,²⁵Phe exendin-4 (1-28) amide [SEQ ID NO 41: His Gly Glu Gly Thr Phe Thr Ser Asp Leu Ser Lys Gln Leu Glu Glu Glu Ala Val Arg Leu Phe Ile Glu Phe Leu Lys Asn-NH₂], and ¹⁴Leu,²² Ala, ²⁵Phe exendin-4 (1-28) amide [SEQ ID NO 8: His Gly Glu Gly Thr Phe Thr Ser Asp Leu Ser Lys Gln Leu Glu Glu Glu Ala Val Arg Leu Ala Ile Glu Phe Leu Lys Asn-NH₂].

In the methods of the present invention, the exendins and exendin agonists may be administered separately or together with one or more other compounds and compositions that exhibit a long-term or short-term triglyceride control action, including, but not limited to other compounds and compositions that comprise a statin, an HMGCoA reductase inhibitor, and/or a triglyceride lowering fibric acid derivative. Suitable statins include, for example, simvastatin, pravastatin, and lovastatin. Suitable triglyceride lowering fibric acid derivatives include gemfibrozil.

### BRIEF DESCRIPTION OF THE DRAWINGS

FIG. 1 depicts the amino acid sequences for certain exendin agonist compounds useful in the present invention [SEQ ID NOS 9-39].

FIG. 2 depicts the mean concentrations of triglyceride in plasma at days 1, 3 and 5 of a clinical study in humans to evaluate the effect of exendin-4 on triglycerides.

### DETAILED DESCRIPTION OF THE INVENTION

Exendins and exendin agonists are useful as described herein in view of their pharmacological properties. As indicated by the human clinical study described in Example 186 below, for example, exendin-4 and agonists thereof will be useful in lowering plasma triglyceride concentrations in ELT subjects, as well as in the treatment of subjects with dyslipidemia (i.e., increased LDL cholesterol, increased VLDL cholesterol, and/or decreased HDL cholesterol).

In the clinical study described in the Examples below, a single-blind, placebo controlled crossover protocol was used to evaluate the effect of multiple doses of synthetic exendin-4 on plasma triglyceride concentrations in people with type 2 diabetes mellitus. The study compared the effects of multiple doses of synthetic exendin-4 and placebo given twice daily (before breakfast and dinner) for five days.

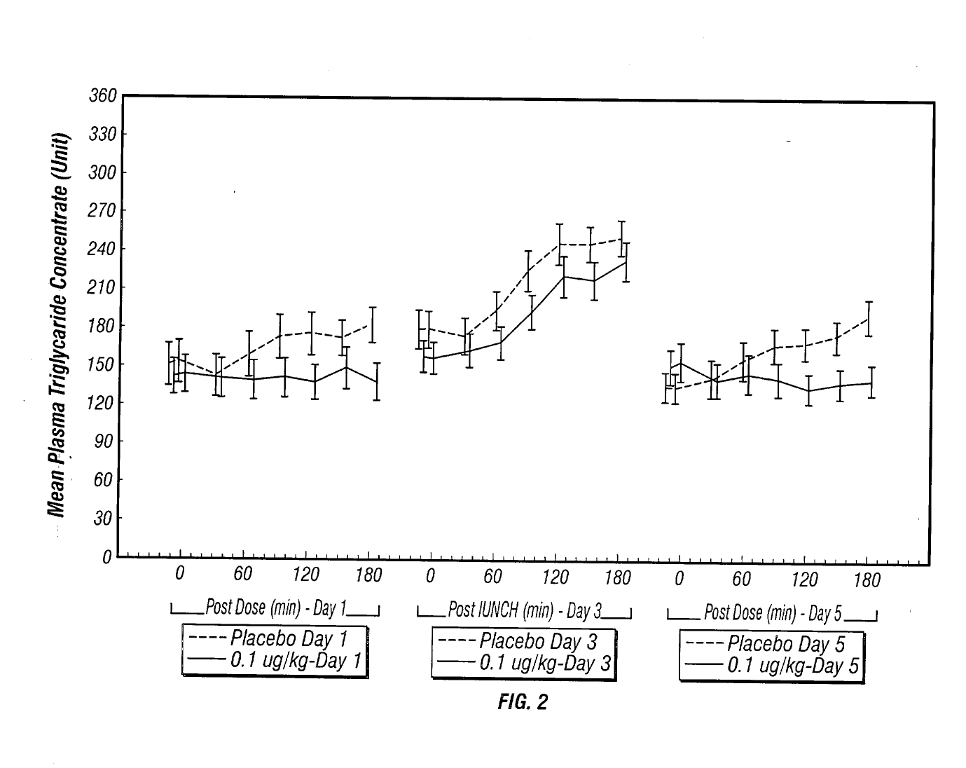

On the mornings of Days 1 and 5, each patient was given a standardized breakfast ten minutes after administration of study medication (placebo or synthetic exendin-4) and blood samples were collected for 3 hours thereafter. Patients who received placebo showed a characteristic rise in serum triglycerides following the meal. Patients who received synthetic exendin-4, however, that rise in serum triglycerides was statistically significantly suppressed. Thus, on Day 5, the peak increase in triglycerides was reduced by 24% (P<0.001) and the total triglyceride area under the three-hour curve was reduced by 15% (P=0.0024). As shown in FIG. 2, similar results were observed on Day 1.

On Day 3 subjects were given a standardized lunch composed of solid food 4.5 hours after administration of study medication and a standardized breakfast. Blood samples were collected for three hours post lunch (i.e., from 4.5 to 7.5 hours after administration of exendin-4 or placebo). Serum triglyceride concentrations increased in response to the lunch. However, the total triglyceride area under the three-hour curve was statistically significantly reduced in those patients who received synthetic exendin-4 as compared to placebo, in this case by approximately 20%. These experiments demonstrate the ability of exendin agonists to lower triglycerides, in particular, postprandial triglycerides, among other things as described and claimed herein.

Activity as exendin agonists can be indicated by activity in assays described in the art. Activity as exendin agonists may also be evaluated by their ability to delay gastric empyting, suppress food intake, or suppress glucagon, as referenced above. Activity as exendin agonists may also be evaluated by their affinity to exendin receptors (United States Provisional Application No.60/166,899, entitled, “High Affinity Exendin Receptors,” filed Nov. 22, 1999, which enjoys common ownership with the present invention and is hereby incorporated by reference). Effects of exendins or exendin agonists in modulating triglyceride levels can be identified, evaluated, or screened for, using methods described or referenced herein, or other methods known in the art for determining effects on plasma triglyceride concentrations.

### Exendin Agonist Compounds

Exendin agonist compounds are those described in International Application No. PCT/US98/16387, filed Aug. 6, 1998, entitled, “Novel Exendin Agonist Compounds,” which claims the benefit of United States Provisional Application No. 60/055,404, filed Aug. 8, 1997, including compounds of the formula (I) [SEQ ID NO. 3]:

Xaa₁ Xaa₂ Xaa₃ Gly Thr Xaa₄ Xaa₅ Xaa₆ Xaa₇ Xaa₈

Ser Lys Gln Xaa₉ Glu Glu Glu Ala Val Arg Leu

Xaa₁₀Xaa₁₁ Xaa₁₂ Xaa₁₃ Leu Lys Asn Gly Gly Xaa₁₄

Ser Ser Gly Ala Xaa₁₅ Xaa₁₆ Xaa₁₇ Xaa₁₈-Z

wherein Xaa₁ is His, Arg or Tyr; Xaa₂ is Ser, Gly, Ala or Thr; Xaa₃ is Asp or Glu; Xaa₄ is Phe, Tyr or naphthylalanine; Xaa₅ is Thr or Ser; Xaa₆ is Ser or Thr; Xaa₇ is Asp or Glu; Xaa₈ is Leu, Ile, Val, pentylglycine or Met; Xaa₉ is Leu, Ile, pentylglycine, Val or Met; Xaa₁₀ is Phe, Tyr or naphthylalanine; Xaa₁₁ is Ile, Val, Leu, pentylglycine, tert-butylglycine or Met; Xaa₁₂ is Glu or Asp; Xaa₁₃ is Trp, Phe, Tyr, or naphthylalanine; Xaa₁₄, Xaa₁₅, Xaa₁₆ and Xaa₁₇ are independently Pro, homoproline, 3Hyp, 4Hyp, thioproline, N-alkylglycine, N-alkylpentylglycine or N-alkylalanine; Xaa₁₈ is Ser, Thr or Tyr; and Z is —OH or —NH₂; with the proviso that the compound is not exendin-3 or exendin-4.

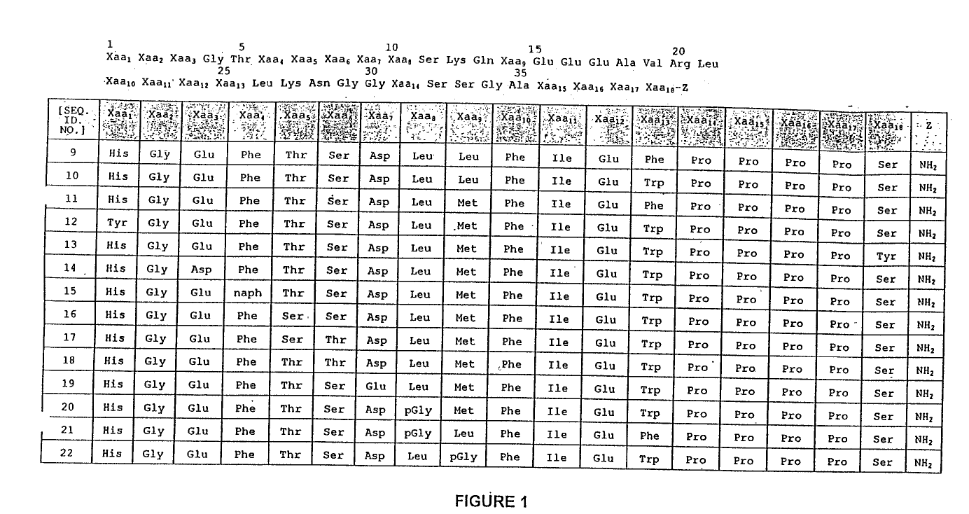

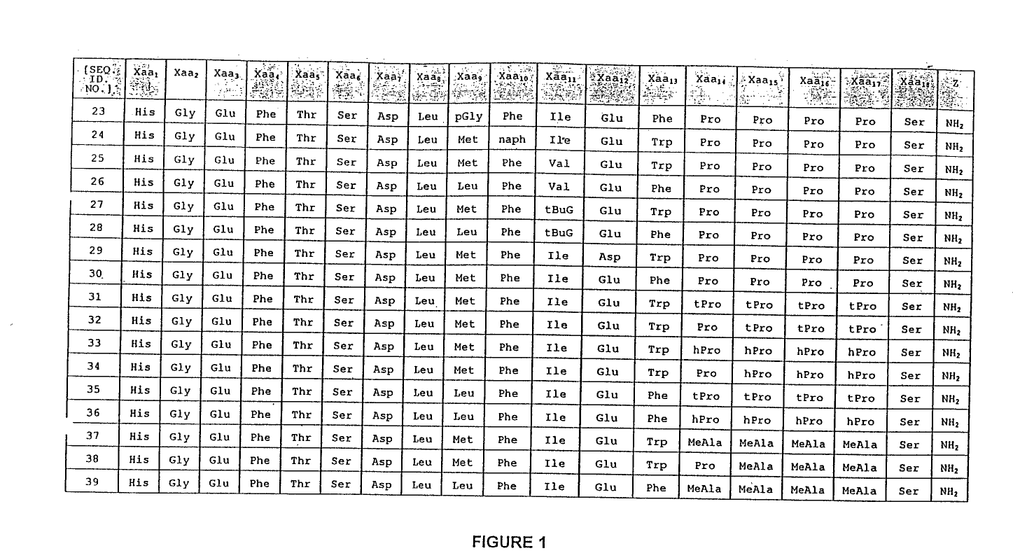

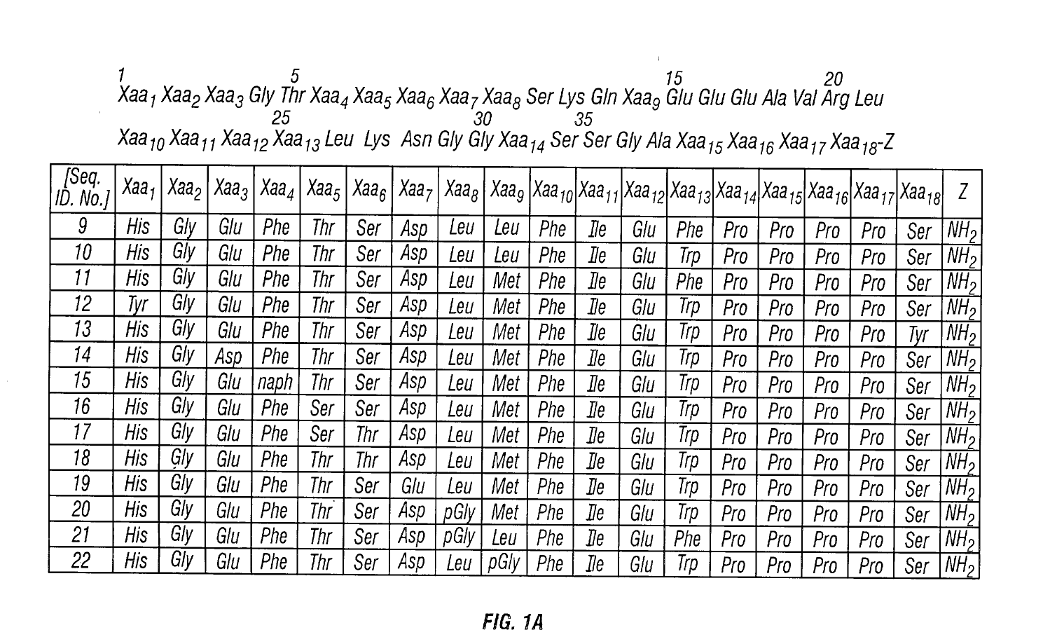

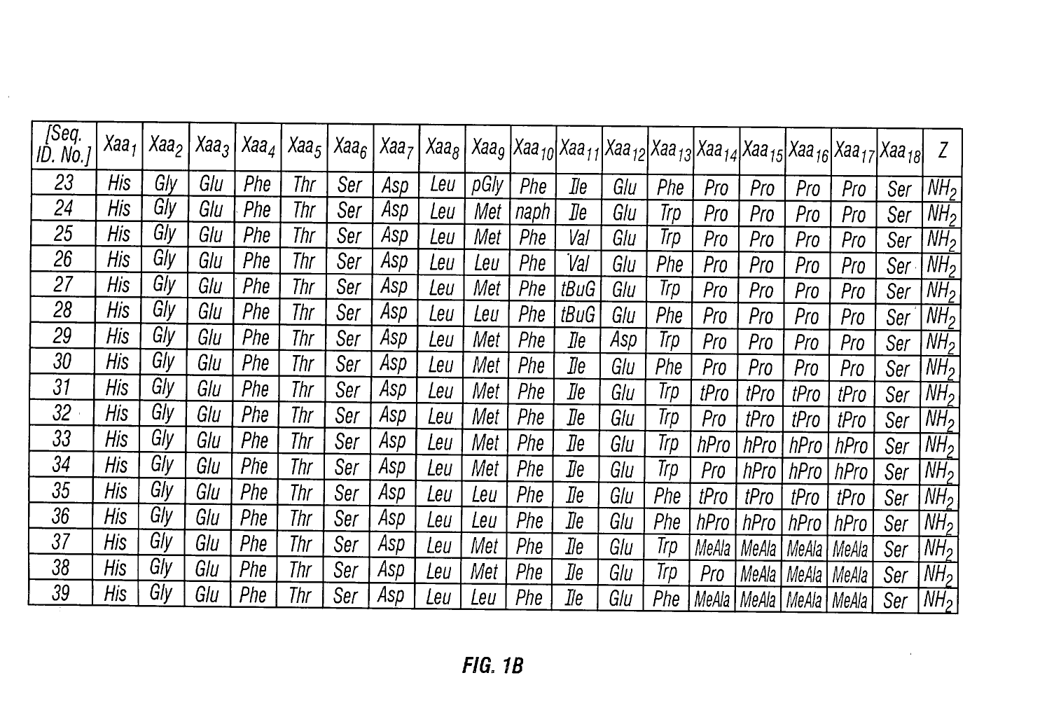

Preferred N-alkyl groups for N-alkylglycine, N-alkylpentylglycine and N-alkylalanine include lower alkyl groups preferably of 1 to about 6 carbon atoms, more preferably of 1 to 4 carbon atoms. Suitable compounds include those listed in FIG. 1 having amino acid sequences of SEQ. ID. NOS. 9 to 39.

Preferred exendin agonist compounds include those wherein Xaa₁ is His or Tyr. More preferably Xaa₁ is His.

Preferred are those compounds wherein Xaa₂ is Gly.

Preferred are those compounds wherein Xaa₉ is Leu, pentylglycine or Met.

Preferred compounds include those wherein Xaa₁₃ is Trp or Phe.

Also preferred are compounds where Xaa₄ is Phe or naphthylalanine; Xaa₁₁ is Ile or Val and Xaa₁₄, Xaa₁₅, Xaa₁₆ and Xaa₁₇ are independently selected from Pro, homoproline, thioproline or N-alkylalanine. Preferably N-alkylalanine has a N-alkyl group of 1 to about 6 carbon atoms.

According to an especially preferred aspect, Xaa₁₅, Xaa₁₆ and Xaa₁₇ are the same amino acid reside.

Preferred are compounds wherein Xaa₁₈ is Ser or Tyr, more preferably Ser.

Preferably Z is —NH₂.

According to one aspect, preferred are compounds of formula (I) wherein Xaa₁ is His or Tyr, more preferably His; Xaa₂ is Gly; Xaa₄ is Phe or naphthylalanine; Xaa₉ is Leu, pentylglycine or Met; Xaa₁₀ is Phe or naphthylalanine; Xaa₁ is Ile or Val; Xaa₁₄, Xaa₁₅, Xaa₁₆ and Xaa₁₇ are independently selected from Pro, homoproline, thioproline or N-alkylalanine; and Xaa₁₈ is Ser or Tyr, more preferably Ser. More preferably Z is —NH₂.

According to an especially preferred aspect, especially preferred compounds include those of formula (I) wherein: Xaa₁ is His or Arg; Xaa₂ is Gly; Xaa₃ is Asp or Glu; Xaa₄ is Phe or napthylalanine; Xaa₅ is Thr or Ser; Xaa₆ is Ser or Thr; Xaa₇ is Asp or Glu;

Xaa₈ is Leu or pentylglycine; Xaa₉ is Leu or pentylglycine; Xaa₁₀ is Phe or naphthylalanine; Xaa₁₁ is Ile, Val or t-butyltylglycine; Xaa₁₂ is Glu or Asp; Xaa₁₃ is Trp or Phe; Xaa₁₄, Xaa₁₅, Xaa₁₆, and Xaa₁₇ are independently Pro, homoproline, thioproline, or N-methylalanine; Xaa₁₈ is Ser or Tyr: and Z is —OH or —NH₂; with the proviso that the compound does not have the formula of either SEQ. ID. NOS. 1 or 2. More preferably Z is —NH₂. Especially preferred compounds include those having the amino acid sequence of SEQ. ID. NOS. 9, 10, 21, 22, 23, 26, 28, 34, 35 and 39.

According to an especially preferred aspect, provided are compounds where Xaa₉ is Leu, Ile, Val or pentylglycine, more preferably Leu or pentylglycine, and Xaa₁₃ is Phe, Tyr or naphthylalanine, more preferably Phe or naphthylalanine. These compounds will exhibit advantageous duration of action and be less subject to oxidative degradation, both in vitro and in vivo, as well as during synthesis of the compound.

Exendin agonist compounds also include those described in International Application No. PCT/US98/24210, filed Nov. 13, 1998, entitled, “Novel Exendin Agonist compounds,” which claims the benefit of United States Provisional Application No. 60/065,442, filed Nov. 14, 1997, including compounds of the formula (II) [SEQ ID NO. 4]:

```
Xaa₁ Xaa₂ Xaa₃ Gly Xaa₅ Xaa₆ Xaa₇ Xaa₈ Xaa₉ Xaa₁₀
Xaa₁₁ Xaa₁₂ Xaa₁₃ Xaa₁₄ Xaa₁₅ Xaa₁₆ Xaa₁₇ Ala Xaa₁₉ Xaa₂₀
Xaa₂₁ Xaa₂₂ Xaa₂₃ Xaa₂₄ Xaa₂₅ Xaa₂₆ Xaa₂₇ Xaa₂₈-Z₁; wherein
Xaa₁ is His, Arg or Tyr;
Xaa₂ is Ser, Gly, Ala or Thr;
Xaa₃ is Asp or Glu;
Xaa₅ is Ala or Thr;
Xaa₆ is Ala, Phe, Tyr or naphthylalanine;
Xaa₇ is Thr or Ser;
Xaa₈ is Ala, Ser or Tbr;
Xaa₉ is Asp or Glu;
Xaa₁₀ is Ala, Leu, Ile, Val, pentylglycine or Met;
Xaa₁₁ is Ala or Ser;
Xaa₁₂ is Ala or Lys;
Xaa₁₃ is Ala or Gln;
Xaa₁₄ is Ala, Leu, Ile, pentylglycine, Val or Met;
Xaa₁₅ is Ala or Glu;
Xaa₁₆ is Ala or Glu;
Xaa₁₇ is Ala or Glu;
Xaa₁₉ is Ala or Val;
Xaa₂₀ is Ala or Mg;
Xaa₂₁ is Ala or Leu;
Xaa₂₂ is Ala, Phe, Tyr or naphthylalanine;
Xaa₂₃ is Ile, Val, Leu, pentylglycine, tert-butylglycine
or Met;
Xaa₂₄ is Ala, Glu or Asp;
Xaa₂₅ is Ala, Trp, Phe, Tyr or naphthylalanine;
Xaa₂₆ is Ala or Leu;
Xaa₂₇ is Ala or Lys;
Xaa₂₈ is Ala or Asn;
Z₁ is—OH,
      —NH2
            Gly-Z₂,
            Gly Gly-Z₂,
            Gly Gly Xaa₃₁ -Z₂,
            Gly Gly Xaa₃₁ Ser-Z₂,
            Gly Gly Xaa₃₁ Ser Ser-Z₂,
            Gly Gly Xaa₃₁ Ser Ser Gly-Z₂,
Gly Gly Xaa₃₁ Ser Ser Gly Ala-Z₂,
Gly Gly Xaa₃₁ Ser Ser Gly Ala Xaa₃₆-Z₂,
Gly Gly Xaa₃₁ Ser Ser Gly Ala Xaa₃₆ Xaa₃₇-Z₂ or
Gly Gly Xaa₃₁ Ser Ser Gly Ala Xaa₃₆ Xaa₃₇ Xaa₃₈-Z₂;
Xaa₃₁, Xaa₃₆, Xaa₃₇ and Xaa₃₈ are independently Pro,
homoproline, 3Hyp, 4Hyp, thioproline,
N-alkylglycine, N-alkylpentylglycine or
N-alkylalanine; and
Z₂ is —OH or —NH₂;
```

provided that no more than three of Xaa₃, Xaa₅, Xaa₆, Xaa₈, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa ₂₈ are Ala.

Preferred N-alkyl groups for N-alkylglycine, N-alkylpentylglycine and N-alkylalanine include lower alkyl groups preferably of 1 to about 6 carbon atoms, more preferably of 1 to 4 carbon atoms.

Preferred exendin agonist compounds include those wherein Xaa₁ is His or Tyr. More preferably Xaa₁ is His.

Preferred are those compounds wherein Xaa₂ is Gly.

Preferred are those compounds wherein Xaa₁₄ is Leu, pentylglycine or Met.

Preferred compounds are those wherein Xaa₂₅ is Trp or Phe.

Preferred compounds are those where Xaa₆ is Phe or naphthylalanine; Xaa₂₂ is Phe or naphthylalanine and Xaa₂₃ is Ile or Val.

Preferred are compounds wherein Xaa₃₁, Xaa₃₆, Xaa₃₇ and Xaa₃₈ are independently selected from Pro, homoproline, thioproline and N-alkylalanine.

Preferably Z, is —NH_{2.}

Preferable Z₂ is —NH₂.

According to one aspect, preferred are compounds of formula (II) wherein Xaa₁ is His or Tyr, more preferably His; Xaa₂ is Gly; Xaa₆ is Phe or naphthylalanine; Xaa₁₄ is Leu, pentylglycine or Met; Xaa₂₂ is Phe or naphthylalanine; Xaa₂₃ is Ile or Val; Xaa₃₁, Xaa₃₆, Xaa₃₇ and Xaa₃₈ are independently selected from Pro, homoproline, thioproline or N-alkylalanine. More preferably Z₁ is —NH₂.

According to an especially preferred aspect, especially preferred compounds include those of formula (II) wherein: Xaa₁ is His or Arg; Xaa₂ is Gly or Ala; Xaa₃ is Asp or Glu; Xaa₅ is Ala or Thr; Xaa₆ is Ala, Phe or nephthylalaine; Xaa₇ is Thr or Ser; Xaa₈ is Ala, Ser or Thr; Xaa₉ is Asp or Glu; Xaa₁₀ is Ala, Leu or pentylglycine; Xaa₁₁ is Ala or Ser; Xaa₁₂ is Ala or Lys; Xaa₁₃ is Ala or Gln; Xaa₁₄ is Ala, Leu or pentylglycine; Xaa₁₅ is Ala or Glu; Xaa₁₆ is Ala or Glu; Xaa₁₇ is Ala or Glu; Xaa₁₉ is Ala or Val; Xaa₂₀ is Ala or Arg; Xaa₂₁ is Ala or Leu; Xaa₂₂ is Phe or naphthylalanine; Xaa₂₃ is Ile, Val or tert-butylglycine; Xaa₂₄ is Ala, Glu or Asp; Xaa₂₅ is Ala, Trp or Phe; Xaa₂₆ is Ala or Leu; Xaa₂₇ is Ala or Lys; Xaa₂₈ is Ala or Asn; Z₁ is —OH, —NH₂, Gly-Z₂, Gly Gly-Z₂, Gly Gly Xaa₃₁-Z₂, Gly Gly Xaa₃₁ Ser-Z₂, Gly Gly Xaa₃ Ser Ser-Z₂, Gly Gly Xaa₃₁ Ser Ser Gly-Z₂, Gly Gly Xaa₃₁Ser Ser Gly Ala-Z₂, Gly Gly Xaa₃₁ Ser Ser Gly Ala Xaa₃₆-Z₂, Gly Gly Xaa₃₁Ser Ser Gly Ala Xaa₃₆ Xaa₃₇-Z₂, Gly Gly Xaa₃₁ Ser Ser Gly Ala Xaa₃₆ Xaa₃₇ Xaa₃₈-Z₂; Xaa₃₁, Xaa₃₆, Xaa₃₇ and Xaa₃₈ being independently Pro homoproline, thioproline or N-methylalanine; and Z₂ being —OH or —NH₂; provided that no more than three of Xaa₃, Xaa₅, Xaa₆, Xaa₈, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala. Especially preferred compounds include those having the amino acid sequence of SEQ. ID. NOS. 40-61.

According to an especially preferred aspect, provided are compounds where Xaa₁₄ is Leu, Ile, Val or pentylglycine, more preferably Leu or pentylglycine, and Xaa₂₅ is Phe, Tyr or naphthylalanine, more preferably Phe or naphthylalanine. These compounds will be less susceptive to oxidative degration, both in vitro and in vivo, as well as during synthesis of the compound.

Exendin agonist compounds also include those described in International Patent Application No. PCT/US98/24273, filed Nov. 13, 1998, entitled, “Novel Exendin Agonist Compounds,” which claims the benefit of United States Provisional Application No. 60/066,029, filed Nov. 14,1997, including compounds of the formula (III)[SEQ ID NO. 5]:

```
Xaa₁ Xaa₂ Xaa₃ Xaa₄ Xaa₅ Xaa₆ Xaa₇ Xaa₈ Xaa₉ Xaa₁₀
Xaa₁₁ Xaa₁₂ Xaa₁₃ Xaa₁₄ Xaa₁₅ Xaa₁₆ Xaa₁₇ Ala Xaa₁₉ Xaa₂₀
Xaa₂₁ Xaa₂₂ Xaa₂₃ Xaa₂₄ Xaa₂₅ Xaa₂₆ Xaa₂₇ Xaa₂₈-Z₁; wherein
Xaa₁ is His, Arg, Tyr, Ala, Norval, Val or Norleu;
Xaa₂ is Ser, Gly, Ala or Thr;
Xaa₃ is Ala, Asp or Glu;
Xaa₄ is Ala, Norval, Val, Norleu or Gly;
Xaa₅ is Ala or Thr;
Xaa₆ is Phe, Tyr or naphthylalanine;
Xaa₇ is Thr or Ser;
Xaa₈ is Ala, Ser or Thr;
Xaa₉ is Ala, Norval, Val, Norleu, Asp or Glu;
Xaa₁₀ is Ala, Leu, Ile, Val, pentylglycine or Met;
Xaa₁₁ is Ala or Ser;
Xaa₁₂ is Ala or Lys;
Xaa₁₃ is Ala or Gln;
Xaa₁₄ is Ala, Leu, Ile, pentylglycine, Val or Met;
Xaa₁₅ is Ala or Glu;
Xaa₁₆ is Ala or Glu;
Xaa₁₇ is Ala or Glu;
Xaa₁₉ is Ala or Val;
Xaa₂₀ is Ala or Arg;
Xaa₂₁ is Ala or Leu;
Xaa₂₂ is Phe, Tyr or naphthylalanine;
Xaa₂₃ is Ile, Val, Leu, pentylglycine, tert-butylglycine or Met;
Xaa₂₄ is Ala, Glu or Asp;
Xaa₂₅ is Ala, Trp, Phe, Tyr or naphthylalanine;
Xaa₂₆ is Ala or Leu;
Xaa₂₇ is Ala or Lys;
Xaa₂₈ is Ala or Asn;
Z₁ is —OH,
                —NH₂,
                Gly-Z₂,
                Gly Gly-Z₂,
                Gly Gly Xaa₃₁-Z₂,
                Gly Gly Xaa₃₁ Ser-Z₂,
                Gly Gly Xaa₃₁ Ser Ser-Z₂,
                Gly Gly Xaa₃₁ Ser Ser Gly-Z₂,
                Gly Gly Xaa₃₁ Ser Ser Gly Ala-Z₂,
                Gly Gly Xaa₃₁ Ser Ser Gly Ala Xaa₃₆-Z₂,
                Gly Gly Xaa₃₁ Ser Ser Gly Ala Xaa₃₆ Xaa₃₇-Z₂,
                Gly Gly Xaa₃₁ Ser Ser Gly Ala Xaa₃₆ Xaa₃₇ Xaa₃₈-Z₂ or Gly Gly Xaa₃₁ Ser Ser Gly
Ala Xaa₃₆ Xaa₃₇ Xaa₃₈ Xaa₃₉-Z₂; wherein
Xaa₃₁, Xaa₃₆, Xaa₃₇ and Xaa₃₈ are independently
Pro, homoproline, 3Hyp, 4Hyp, thioproline,
N-alkylglycine, N-alkylpentylglycine or
N-alkylalanine; and
Z₂ is —OH or —NH₂;
```

provided that no more than three of Xaa₃, Xaa₄, Xaa₅, Xa₆, Xaa₈, Xaa₉, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa ₂₆, Xaa₂₇ and Xaa₂₈ are Ala; and provided also that, if Xaa₁ is His, Arg or Tyr, then at least one of Xaa₃, Xaa₄ and Xaa₉ is Ala.

### Definitions

In accordance with the present invention and as used herein, the following terms are defined to have the following meanings, unless explicitly stated otherwise.

The term “amino acid” refers to natural amino acids, unnatural amino acids, and amino acid analogs, all in their D and L stereoisomers if their structure allow such stereoisomeric forms. Natural amino acids include alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), Lysine (Lys), methionine (Met), phenylalanine (Phe), proline (Pro), serine (Ser), threonine (Thr), typtophan (Trp), tyrosine (Tyr) and valine (Val). Unnatural amino acids include, but are not limited to azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic, acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, norleucine, omithine, pentylglycine, pipecolic acid and thioproline. Amino acid analogs include the natural and unnatural amino acids which are chemically blocked, reversibly or irreversibly, or modified on their N-terminal amino group or their side-chain groups, as for example, methionine sulfoxide, methionine sulfone, S-(carboxymethyl)-cysteine, S-(carboxymethyl)-cysteine sulfoxide and S-(carboxymethyl)-cysteine sulfone.

The term “amino acid analog” refers to an amino acid wherein either the C-terminal carboxy group, the N-terminal amino group or side-chain functional group has been chemically codified to another functional group. For example, aspartic acid-(beta-methyl ester) is an amino acid analog of aspartic acid; N-ethylglycine is an amino acid analog of glycine; or alanine carboxamide is an amino acid analog of alanine.

The term “amino acid residue” refers to radicals having the structure: (1) —C(O)—R—NH—, wherein R typically is —CH(R′)—, wherein R′ is an amino acid side chain, typically H or a carbon containing substitutent; or (2)

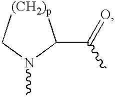

wherein p is 1, 2 or 3 representing the azetidinecarboxylic acid, proline or pipecolic acid residues, respectively.

The term “lower” referred to herein in connection with organic radicals such as alkyl groups defines such groups with up to and including about 6, preferably up to and including 4 and advantageously one or two carbon atoms. Such groups may be straight chain or branched chain.

“Pharmaceutically acceptable salt” includes salts of the compounds described herein derived from the combination of such compounds and an organic or inorganic acid. In practice, the use of the salt form amounts to use of the base form. The compounds are useful in both free base and salt form.

In addition, the following abbreviations stand for the following:

“ACN” or “CH₃CN” refers to acetonitrile.

“Boc”, “tboc” or “Tboc” refers to t-butoxy carbonyl.

“DCC” refers to N,N′-dicyclohexylcarbodiimide.

“Fmoc” refers to fluorenylmethoxycarbonyl.

“HBTU”refers to 2-(1H-benzotriazol-1-yl)-1,1,3,3,-tetramethyluronium hexaflurophosphate.

“HOBt” refers to 1-hydroxybenzotriazole monohydrate.

“homoP” or hpro” refers to homoproline.

“MeAla” or “Nme” refers to N-methylalanine.

“naph” refers to naphthylalanine.

“pG” or pGly” refers to pentylglycine.

“tBuG” refers to tertiary-butylglycine.

“ThioP” or tPro” refers to thioproline.

3Hyp” refers to 3-hydroxyproline

4Hyp” refers to 4-hydroxyproline

NAG” refers to N-alkylglycine

NAPG” refers to N-alkylpentylglycine

“Norval” refers to norvaline

“Norleu” refers to norleucine

### Preiparation of Compounds

The exendins and exendin agonists described herein may be prepared using standard solid-phase peptide synthesis techniques and preferably an automated or semiautomated peptide synthesizer. Typically, using such techniques, an α-N-carbamoyl protected amino acid and an amino acid attached to the growing peptide chain on a resin are coupled at room temperature in an inert solvent such as dimethylformamide, N-methylpyrrolidinone or methylene chloride in the presence of coupling agents such as dicyclohexylcarbodiimide and 1-hydroxybenzotriazole in the presence of a base such as diisopropylethylamine. The α-N-carbamoyl protecting group is removed from the resulting peptide-resin using a reagent such as trifluoroacetic acid or piperidine, and the coupling reaction repeated with the next desired N-protected amino acid to be added to the peptide chain. Suitable N-protecting groups are well known in the art, with t-butyloxycarbonyl (tBoc) and fluorenylmethoxycarbonyl (Fmoc) being preferred herein.

The solvents, amino acid derivatives, and 4-methylbenzhydryl-amine resin used in the peptide synthesizer may be purchased from Applied Biosystems Inc. (Foster City, Calif.). The following side-chain protected amino acids may be purchased from Applied Biosystems, Inc.: Boc-Arg(Mts), Fmoc-Arg(Pmc), Boc-Thr(Bzl), Fmoc-Thr(t-Bu), Boc-Ser(Bzl), Fmoc-Ser(t-Bu), Boc-Tyr(BrZ), Fmoc-Tyr(t-Bu), Boc-Lys(Cl-Z), Fmoc-Lys(Boc), Boc-Glu(Bzl), Fmoc-Glu(t-Bu), Fmoc-His(Trt), Fmoc-Asn(Trt), and Fmoc-Gln(Trt). Boc-His(BOM) may be purchased from Applied Biosystems, Inc. or Bachem Inc. (Torrance, Calif.). Anisole, dimethylsulfide, phenol, ethanedithiol, and thioanisole may be obtained from Aldrich Chemical Company (Milwaukee, Wis.). Air Products and Chemicals (Allentown, Pa.) supplies HF. Ethyl ether, acetic acid and methanol may be purchased from Fisher Scientific (Pittsburgh, Pa).

Solid phase peptide synthesis may be carried out with an automatic peptide synthesizer (Model 430A, Applied Biosystems Inc., Foster City, Calif.) using the NMP/HOBt (Option 1) system and tBoc or Fmoc chemistry (see, Applied Biosystems User's Manual for the ABI 430A Peptide Synthesizer, Version 1.3B Jul. 1, 1988, section 6, pp. 49-70, Applied Biosystems, Inc., Foster City, Calif.) with capping. Boc-peptide-resins may be cleaved with HF (−5° C. to 0° C., 1 hour). The peptide may be extracted from the resin with alternating water and acetic acid, and the filtrates lyophilized. The Fmoc-peptide resins may be cleaved according to standard methods (Introduction to Cleavage Techniques, Applied Biosystems, Inc., 1990, pp. 6-12). Peptides may be also be assembled using an Advanced Chem Tech Synthesizer (Model MPS 350, Louisville, Ky.).

Peptides may be purified by RP-HPLC (preparative and analytical) using a Waters Delta Prep 3000 system. A C4, C8 or C18 preparative column (10 μ, 2.2×25 cm; Vydac, Hesperia, Calif.) may be used to isolate peptides, and purity may be determined using a C4, C8 or C18 analytical column (5 μ, 0.46×25 cm; Vydac). Solvents (A=0.1% TFA/water and B=0.1% TFA/CH₃CN) may be delivered to the analytical column at a flowrate of 1.0 ml/min and to the preparative column at 15 ml/min. Amino acid analyses may be performed on the Waters Pico Tag system and processed using the Maxima program. Peptides may be hydrolyzed by vapor-phase acid hydrolysis (115° C., 20-24 h). Hydrolysates may be derivatized and analyzed by standard methods (Cohen, et al., The Pico Tag Method: A Manual of Advanced Techniques for Amino Acid Analysis, pp. 11-52, Millipore Corporation, Milford, Mass. (1989)). Fast atom bombardment analysis may be carried out by M-Scan, Incorporated (West Chester, Pa.). Mass calibration may be performed using cesium iodide or cesium iodide/glycerol. Plasma desorption ionization analysis using time of flight detection may be carried out on an Applied Biosystems Bio-Ion 20 mass spectrometer. Electrospray mass spectroscopy may be carried out on a VG-Trio machine.

Peptide compounds useful in the invention may also be prepared using recombinant DNA techniques, using methods now known in the art. See, e.g., Sambrook et al., Molecular Cloning: A Laboratory Manual, 2d Ed., Cold Spring Harbor (1989). Non-peptide compounds useful in the present invention may be prepared by art-known methods. For example, phosphate-containing amino acids and peptides containing such amino acids may be prepared using methods known in the art. See, e.g., Bartlett and Landen, Biorg. Chem. 14:356-377 (1986).

Compositions useful in the invention may conveniently be provided in the form of formulations suitable for parenteral (including intravenous, intramuscular, and subcutaneous) or nasal or oral administration. In some cases, it will be convenient to provide an exendin or exendin agonist and another lipid-controlling agent, such as a statin, in a single composition or solution for administration together. In other cases, it may be more advantageous to administer the additional agent separately from said exendin or exendin agonist. A suitable administration format may best be determined by a medical practitioner for each patient individually. Suitable pharmaceutically acceptable carriers and their formulation are described in standard formulation treatises, e.g., Remington's Pharmaceutical Sciences by E. W. Martin. See also Wang, Y. J. and Hanson, M. A. “Parenteral Formulations of Proteins and Peptides: Stability and Stabilizers,” Journal of Parenteral Science and Technology, Technical Report No.10, Supp. 42:2S (1988).

Compounds useful in the invention can be provided as parenteral compositions for injection or infusion. Preferred formulations are those described and claimed in U.S. application Ser. No. 60/116,380, entitled, “Novel Exendin Agonist Formulations and Methods of Administration Thereof,” filed Jan. 14, 1999, which enjoys common ownership with the present application and which is incorporated by this reference into the present application as though fully set forth herein.

Formulations include, for example, compounds suspended in an inert oil, suitably a vegetable oil such as sesame, peanut, olive oil, or other acceptable carrier. Preferably, they are suspended in an aqueous carrier, for example, in an isotonic buffer solution at a pH of about 3.0 to 8.0, preferably at a pH of about 3.5 to 5.0. These compositions may be sterilized by conventional sterilization techniques, or may be sterile filtered. The compositions may contain pharmaceutically acceptable auxiliary substances as required to approximate physiological conditions, such as pH buffering agents. Useful buffers include for example, sodium acetate/acetic acid buffers. Formulations may also include a preservative. A preferred preservative is m-cresol, preferably 0.3% m-cresol. A form of repository or “depot” slow release preparation may be used so that therapeutically effective amounts of the preparation are delivered into the bloodstream over many hours or days following transdermal injection or delivery.

The desired isotonicity may be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions.

The claimed compositions can also be formulated as pharmaceutically acceptable salts (e.g., acid addition salts) and/or complexes thereof. Pharmaceutically acceptable salts are non-toxic salts at the concentration at which they are administered. The preparation of such salts can facilitate the pharmacological use by altering the physical-chemical characteristics of the composition without preventing the composition from exerting its physiological effect. Examples of useful alterations in physical properties include lowering the melting point to facilitate transmucosal administration and increasing the solubility to facilitate the administration of higher concentrations of the drug.

Pharmaceutically acceptable salts include acid addition salts such as those containing sulfate, hydrochloride, phosphate, sulfamate, acetate, citrate, lactate, tartrate, methanesulfonate, ethanesulfonate, benzenesulfonate, p-toluenesulfonate, cyclohexylsulfamate and quinate. Acetate salts are preferred. Pharmaceutically acceptable salts can be obtained from acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid. Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

Carriers or excipients can also be used to facilitate administration of the compound. Examples of carriers and excipients include calcium carbonate, calcium phosphate, various sugars such as lactose, glucose, or sucrose, or types of starch, cellulose derivatives, gelatin, vegetable oils, polyethylene glycols and physiologically compatible solvents. The compositions or pharmaceutical composition can be administered by different routes including intravenously, intraperitoneal, subcutaneous, and intramuscular, orally, topically, transmucosally, or by pulmonary inhalation. Preferred methods of administration are those described and claimed in U.S. Application Serial No. 60/116,380, entitled, “Novel Exendin Agonist Formulations and Methods of Administration Thereof,” filed Jan. 14, 1999, which has been incorporated by reference into this application.

If desired, solutions of the above compositions may be thickened with a thickening agent such as methylcellulose. They may be prepared in emulsified form, either water in oil or oil in water. Any of a wide variety of pharmaceutically acceptable emulsifying agents may be employed including, for example, acacia powder, a non-ionic surfactant (such as a Tween), or an ionic surfactant (such as alkali polyether alcohol sulfates or sulfonates, eg., a Triton).

Compositions useful in the invention are prepared by mixing the ingredients following generally accepted procedures. For example, the selected components may be simply mixed in a blender or other standard device to produce a concentrated mixture which may then be adjusted to the final concentration and viscosity by the addition of water or thickening agent and possibly a buffer to control pH or an additional solute to control tonicity.

For use by the physician, the compositions will be provided in dosage unit form containing an amount of an exendin or exendin agonist, for example, exendin-3, and/or exendin-4, with or without another triglyceride-lowering agent. Therapeutically effective amounts of an exendin or exendin agonist for use treating a subject with elevated triglyceride levels are those that lower triglycerides to a desired level. As will be recognized by those in the field, an effective amount of therapeutic agent will vary with many factors including the age and weight of the patient, the patient's physical condition, the blood triglyceride level and other factors.

The effective daily plasma triglyceride controlling dose of the compounds will typically be in the range of from about 0.5-3 to 20-30 μg to about 1 mg/day and, more specifically, from about 1-20 μg to about 500 μg/day for a 70 kg patient, administered in a single or divided doses. Still more specifically, the effective daily plasma triglyceride controlling dose of the compounds will typically be in the range of from about about 1-20 μg to about 100 μg/day and, more specifically about 1-3 μg to about 20-50 μg/day, for a 70 kg patient, administered in a single or divided doses.

Various preferred dosages are described in U.S. Application Serial No. 60/116,380, entitled, “Novel Exendin Agonist Formulations and Methods of Administration Thereof,” filed Jan. 14, 1999, which has been incorporated by reference into the present application.

A preferred dose for twice daily administration of is about 0.01-0.05 to about 0.1-0.3 μg per kilogram. Preferred doses based upon patient weight for compounds having approximately the potency of exendin-4 range from 0.005 μg/kg per dose to about 0.2 μg/kg per dose. More preferably, doses based upon patient weight for compounds having approximately the potency of exendin-4 range from 0.02 μg/kg per dose to about 0.1 μg/kg per dose. Most preferrably, doses based upon patient weight for compounds having approximately the potency of exendin-4 range from 0.05 μg/kg per dose to about 0.1 μg/kg per dose. These doses are administered from 1 to 4 times per day, preferably from 1 to 2 times per day. Doses of exendins or exendin agonist will normally be less if given by continuous infusion.

The exact dose to be administered is determined by the attending clinician and is dependent upon where the particular compound lies within the above quoted range, as well as upon the age, weight and condition of the individual, and the mode of adminstration. Administration should begin shortly after diagnosis of elevated triglycerides (or other dyslipidemia) and continue for until the desired triglyceride (or other lipid) level is reached. Administration may be by injection, preferably subcutaneous or intramuscular. Administration may also be by non-injectable routes, for example, via the respiratory tract, the mouth, and the gut. Orally active compounds may be taken orally, however dosages should be increased 5-10 fold. Solid dosage forms, such as those useful for oral, buccal, sublingual, intra-tracheal, nasal or pulmonary delivery may be used. Additionally, preserved or unpreserved liquid formulations or dry powder may be used.

The optimal formulation and mode of administration of compounds of the present application to a patient depend on factors known in the art such as the disease or disorder associated with elevated triglyceride levels, dyslipidemia, the desired effect, and the type of patient. While the compounds will typically be used to treat human subjects they may also be used to treat similar or identical conditions in other vertebrates such as other primates, farm animals such as swine, cattle and poultry, and sports animals and pets such as horses, dogs and cats.

To assist in understanding the present invention, the following Examples are included. The experiments relating to this invention should not, of course, be construed as specifically limiting the invention and such variations of the invention, now known or later developed, which would be within the purview of one skilled in the art are considered to fall within the scope of the invention as described herein and hereinafter claimed.

### EXAMPLE 1

### Preparation of Amidated Epptide Having SEQ. ID. NO. 9

The above-identified peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.). In general, single-coupling cycles were used throughout the synthesis and Fast Moc (HBTU activation) chemistry was employed. However, at some positions coupling was less efficient than expected and double couplings were required. In particular, residues Asp₉, Thr₇ and Phe₆ all required double coupling. Deprotection (Fmoc group removal) of the growing peptide chain using piperidine was not always efficient. Double deprotection was required at positions Arg₂₀, Val₁₉ and Leu₁₄. Final deprotection of the completed peptide resin was achieved using a mixture of triethylsilane (0.2 mL), ethanedithiol (0.2 mL), anisole (0.2 mL), water (0.2 mL) and trifluoroacetic acid (15 mL) according to standard methods (Introduction to Cleavage Techniques, Applied Biosystems, Inc.) The peptide was precipitated in ether/water (50 mL) and centrifuged. The precipitate was reconstituted in glacial acetic acid and lyophilized. The lyophilized peptide was dissolved in water). Crude purity was about 55%.

Used in purification steps and analysis were Solvent A (0.1% TFA in water) and Solvent B (0.1% TFA in ACN).

The solution containing peptide was applied to a preparative C-18 column and purified (10% to 40% Solvent B in Solvent A over 40 minutes). Purity of fractions was determined isocratically using a C-18 analytical column. Pure fractions were pooled furnishing the above-identified peptide. Analytical RP-HPLC (gradient 30% to 60% Solvent B in Solvent A over 30 minutes) of the lyophilized peptide gave product peptide having an observed retention time of 14.5 minutes. Electrospray Mass Spectrometry (M): calculated 4131.7; found 4129.3.

### EXAMPLE 2

### Preparation of Peptide Having SEQ. ID. NO. 10

The above-identified peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis were Solvent A (0.1% TFA in water) and Solvent B (0.1% TFA in ACN). Analytical RP-HPLC (gradient 25% to 75% Solvent B in Solvent A over 30 minutes) of the lyophilized peptide gave product peptide having an observed retention time of 21.5 minutes. Electrospray Mass Spectrometry (M): calculated 4168.6; found 4171.2.

### EXAMPLE 3a

### Preparation of Peptide Having SEQ. ID. NO. 11

The above-identified peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis were Solvent A (0.1% TFA in water) and Solvent B (0.1% TFA in ACN). Analytical RP-HPLC (gradient 30% to 60% Solvent B in Solvent A over 30 minutes) of the lyophilized peptide gave product peptide having an observed retention time of 17.9 minutes. Electrospray Mass Spectrometry (M): calculated 4147.6; found 4150.2.

### EXAMPLE 3b

### Preparation of Peptide Having SEQ. ID. NO. 12

The above-identified peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis were Solvent A (0.1% TFA in water) and Solvent B (0.1% TFA in ACN). Analytical RP-HPLC (gradient 35% to 65% Solvent B in Solvent A over 30 minutes) of the lyophilized peptide gave product peptide having an observed retention time of 19.7 minutes. Electrospray Mass Spectrometry (M): calculated 4212.6; found 4213.2.

### EXAMPLE 4

### Preparation of Peptide Having SEQ. ID. NO. 13

The above-identified peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis were Solvent A (0.1% TFA in water) and Solvent B (0.1% TFA in ACN). Analytical RP-HPLC (gradient 30% to 50% Solvent B in Solvent A over 30 minutes) of the lyophilized peptide gave product peptide having an observed retention time of 16.3 minutes. Electrospray Mass Spectrometry (M): calculated 4262.7; found 4262.4.

### EXAMPLE 5

### Preparation of Peptide Having SEQ. ID. NO. 14

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4172.6

### EXAMPLE 6

### Preparation of Peptide Having SEQ. ID. NO. 15

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4224.7.

### EXAMPLE 7

### Preparation of Peptide Having SEQ. ID. NO. 16

The above-identified peptide is assembled on 4-(2′-4′-dimethoxyphenyl)-Fmoc aininomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4172.6

### EXAMPLE 8

### Preparation of Peptide Having SEQ. ID. NO. 17

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4186.6

### EXAMPLE 9

### Preparation of Peptide Having SEQ. ID. NO. 18

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4200.7.

### EXAMPLE 10

### Preparation of Peptide Having SEQ. ID. NO. 19

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4200.7.

### EXAMPLE 11

### Preparation of Peptide Having SEQ. ID. NO. 20

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4202.7.

### EXAMPLE 12

### Preparation of Peptide Having SEQ. ID. NO. 21

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4145.6.

### EXAMPLE 13

### Preparation of Peptide Having SEQ. ID. NO. 22

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis are Solvent A (0. 1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4184.6.

### EXAMPLE 14

### Preparation of Peptide Having SEQ. ID. NO. 23

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4145.6.

### EXAMPLE 15

### Preparation of Peptide Having SEQ. ID. NO. 24

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4224.7.

### EXAMPLE 16

### Preparation of Peptide Having SEQ. ID. NO. 25

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4172.6.

### EXAMPLE 17

### Preparation of Peptide Having SEQ. ID. NO. 26

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis are Solvent A (0. 1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4115.5.

### EXAMPLE 18

### Preparation of Peptide Having SEQ. ID. NO. 27

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4188.6.

### EXAMPLE 19

### Preparation of Peptide Having SEQ. ID. NO. 28

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4131.6.

### EXAMPLE 20

### Preparation of Peptide Having SEQ. ID. NO. 29

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4172.6.

### EXAMPLE 21

### Preparation of Peptide Having SEQ. ID. NO. 30

The above-identified peptide is assembled on 4-(2′-4′-dimethoxyphenyl)-Fmoc arninomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4145.6.

### EXAMPLE 22

### Preparation of Peptide Having SEQ. ID. NO. 31

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Additional double couplings are required at the thioproline positions 38, 37, 36 and 31. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4266.8.

### EXAMPLE 23

### Preparation of Peptide Having SEQ. ID. NO. 32

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Additional double couplings are required at the thioproline positions 38, 37 and 36. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4246.8.

### EXAMPLE 24

### Preparation of Peptide Having SEQ. ID. NO.33

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Additional double couplings are required at the homoproline positions 38, 37, 36 and 31. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4250.8.

### EXAMPLE 25

### Preparation of Peptide Having SEQ. ID. NO. 34

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Additional double couplings are required at the homoproline positions 38, 37, and 36. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4234.8.

### EXAMPLE 26

### Preparation of Peptide Having SEQ. ID. NO. 35

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Additional double couplings are required at the thioproline positions 38, 37, 36 and 31. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4209.8.

### EXAMPLE 27

### Preparation of Peptide Having SEQ. ID. NO. 36

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Additional double couplings are required at the homoproline positions 38, 37, 36 and 31. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4193.7.

### EXAMPLE 28

### Preparation of Peptide Having SEQ. ID. NO.37

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Additional double couplings are required at the N-methylalanine positions 38, 37, 36 and 31. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3858.2.

### EXAMPLE 29

### Preparation of Peptide having SEQ. ID. NO. 38

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Additional double couplings are required at the N-methylalanine positions 38, 37 and 36. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3940.3.

### EXAMPLE 30

### Preparation of Peptide Having SEQ. ID. NO.39

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 1. Additional double couplings are required at the N-methylalanine positions 38, 37, 36 and 31. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3801.1.

### EXAMPLE 31

### Preparation of C-terminal Carboxylic Acid Peptides Corresponding to the Above C-terminal Amide Sequences.

The above peptides of Examples 1 to 30 are assembled on the so called Wang resin (p-alkoxybenzylalacohol resin (Bachem, 0.54 mmole/g)) using Fmoc-protected amino acids (Applied Biosystems, Inc.), cleaved from the resin, deprotected and purified in a similar way to Example 1. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry provides an experimentally determined (M).

### EXAMPLE 32

### Preparation of Peptide Having SEQ ID NO. 7

His Gly Glu Gly Thr Phe Thr Ser Asp Leu Ser Lys Gln Met Glu Glu Glu Ala Val Arg Leu Phe Ile Glu Trp Leu Lys Asn Gly Gly-NH₂ [SEQ. ID. NO. 7]

The above amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.). In general, single-coupling cycles were used throughout the synthesis and Fast Moc (HBTU activation) chemistry was employed. Deprotection (Fmoc group removal) of the growing peptide chain was achieved using piperidine. Final deprotection of the completed peptide resin was achieved using a mixture of triethylsilane (0.2 mL), ethanedithiol (0.2 mL), anisole (0.2 mL), water (0.2 mL) and trifluoroacetic acid (15 mL) according to standard methods (Introduction to Cleavage Techniques, Applied Biosystems, Inc.) The peptide was precipitated in ether/water (50 mL) and centrifuged. The precipitate was reconstituted in glacial acetic acid and lyophilized. The lyophilized peptide was dissolved in water). Crude purity was about 75%.

Used in purification steps and analysis were Solvent A (0.1% TFA in water) and Solvent B (0.1% TFA in ACN). The solution containing peptide was applied to a preparative C-18 column and purified (10% to 40% Solvent B in Solvent A over 40 minutes). Purity of fractions was determined isocratically using a C-18 analytical column. Pure fractions were pooled furnishing the above-identified peptide. Analytical RP-HPLC (gradient 30% to 50% Solvent B in Solvent A over 30 minutes) of the lyophilized peptide gave product peptide having an observed retention time of 18.9 minutes. Electrospray Mass Spectrometry (M): calculated 3408.0; found 3408.9.

### EXAMPLE 33

### Preparation of Peptide Having SEQ ID NO. 40

His Gly Glu Gly Thr Phe Thr Ser Asp Leu Ser Lys Gln Met Glu Glu Glu Ala Val Arg Leu Phe Ile Glu Trp Leu Lys Asn-NH₂ [SEQ. ID. NO. 40]

The above amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 30% to 40% Solvent B in Solvent A over 30 minutes) of the lyophilized peptide gave product peptide having an observed retention time of 17.9 minutes. Electrospray Mass Spectrometry (M): calculated 3294.7; found 3294.8.

### EXAMPLE 34

### Preparation of Peptide Having SEQ ID NO. 41

His Gly Glu Gly Thr Phe Thr Ser Asp Leu Ser Lys Gln Leu Glu Glu Glu Ala Val Arg Leu Phe Ile Glu Phe Leu Lys Asn-NH₂ [SEQ. ID. NO. 41]

The above-identified amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 29% to 36% Solvent B in Solvent A over 30 minutes) of the lyophilized peptide gave product peptide having an observed retention time of 20.7 minutes. Electrospray Mass Spectrometry (M): calculated 3237.6; found 3240.

### EXAMPLE 35

### Preparation of Peptide Having SEQ ID NO. 42

His Ala Glu Gly Thr Phe Thr Ser Asp Leu Ser Lys Gln Leu Glu Glu Glu Ala Val Arg Leu Phe Ile Glu Phe Leu Lys Asn-NH₂ [SEQ. ID. NO. 42]

The above amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 36% to 46% Solvent B in Solvent A over 30 minutes) of the lyophilized peptide gave product peptide having an observed retention time of 15.2 minutes. Electrospray Mass Spectrometry (M): calculated 3251.6; found 3251.5.

### EXAMPLE 36

### Preparation of Peptide Having SEQ ID NO. 43

His Gly Glu Gly Ala Phe Thr Ser Asp Leu Ser Lys Gln Leu Glu Glu Glu Ala Val Arg Leu Phe Ile Glu Phe Leu Lys Asn-NH₂ [SEQ. ID. NO. 43]

The above amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 36% to 46% Solvent B in Solvent A over 30 minutes) of the lyophilized peptide gave product peptide having an observed retention time of 13.1 minutes. Electrospray Mass Spectrometry (M): calculated 3207.6; found 3208.3.

### EXAMPLE 37

### Preparation of Peptide Having SEQ ID NO. 44

His Gly Glu Gly Thr Ala Thr Ser Asp Leu Ser Lys Gln Leu Glu Glu Glu Ala Val Arg Leu Phe Ile Glu Phe Leu Lys Asn-NH₂ [SEQ. ID. NO. 44]

The above amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 35% to 45% Solvent B in Solvent A over 30 minutes) of the lyophilized peptide gave product peptide having an observed retention time of 12.8 minutes. Electrospray Mass Spectrometry (M): calculated 3161.5; found 3163.

### EXAMPLE 38

### Preparation of Peptide Having SEQ II) NO. 45

His Gly Glu Gly Thr Phe Thr Ala Asp Leu Ser Lys Gln Leu Glu Glu Glu Ala Val Arg Leu Phe Ile Glu Phe Leu Lys Asn-NH₂ [SEQ. ID. NO. 45]

The above-identified amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 36% to 46% Solvent B in Solvent A over 30 minutes) of the lyophilized peptide gave product peptide having an observed retention time of 15.2 minutes. Electrospray Mass Spectrometry (M): calculated 3221.6; found 3222.7.

### EXAMPLE 39

### Preparation of Peptide Having SEQ ID NO. 46

His Gly Glu Gly Thr Phe Thr Ser Asp Ala Ser Lys Gln Leu Glu Glu Glu Ala Val Arg Leu Phe Ile Glu Phe Leu Lys Asn-NH₂ [SEQ. ID. NO. 46]

The above-identified amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 34% to 44% Solvent B in Solvent A over 30 minutes) of the lyophilized peptide gave product peptide having an observed retention time of 14.3 minutes. Electrospray Mass Spectrometry (M): calculated 3195.5; found 3199.4.

### EXAMPLE 40

### Preparation of Peptide Having SEQ ID NO. 47

His Gly Glu Gly Thr Phe Thr Ser Asp Leu Ala Lys Gln Leu Glu Glu Glu Ala Val Arg Leu Phe Ile Glu Phe Leu Lys Asn-NH₂ [SEQ. ID. NO. 47]

The above-identified amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 38% to 48% Solvent B in Solvent A over 30 minutes) of the lyophilized peptide gave product peptide having an observed retention time of 15.7 minutes. Electrospray Mass Spectrometry (M): calculated 3221.6; found 3221.6.

### EXAMPLE 41

### Preparation of Peptide Having SEQ ID NO. 48

His Gly Glu Gly Thr Phe Thr Ser Asp Leu Ser Ala Gln Leu Glu Glu Glu Ala Val Arg Leu Phe Ile Glu Phe Leu Lys Asn-NH₂ [SEQ. ID. NO. 48]

The above-identified amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 38% to 48% Solvent B in Solvent A over 30 minutes) of the lyophilized peptide gave product peptide having an observed retention time of 18.1 minutes. Electrospray Mass Spectrometry (M): calculated 3180.5; found 3180.9.

### EXAMPLE 42

### Preparation of Peptide Having SEQ ID NO. 49

His Gly Glu Gly Thr Phe Thr Ser Asp Leu Ser Lys Ala Leu Glu Glu Glu Ala Val Arg Leu Phe Ile Glu Phe Leu Lys Asn-NH₂ [SEQ. ID. NO. 49]

The above-identified amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 36% to 46% Solvent B in Solvent A over 30 minutes) of the lyophilized peptide gave product peptide having an observed retention time of 17.0 minutes. Electrospray Mass Spectrometry (M): calculated 3180.6; found 3182.8.

### EXAMPLE 43

### Preparation of Peptide Having SEQ ID NO. 50

His Gly Glu Gly Thr Phe Thr Ser Asp Leu Ser Lys Gln Ala Glu Glu Glu Ala Val Arg Leu Phe Ile Glu Phe Leu Lys Asn-NH₂ [SEQ. ID. NO. 50]

The above-identified amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 32% to 42% Solvent B in Solvent A over 30 minutes) of the lyophilized peptide gave product peptide having an observed retention time of 14.9 minutes. Electrospray Mass Spectrometry (M): calculated 3195.5; found 3195.9.

### EXAMPLE 44

### Preparation of Peptide Having SEQ ID NO. 51

His Gly Glu Gly Thr Phe Thr Ser Asp Leu Ser Lys Gln Leu Ala Glu Glu Ala Val Arg Leu Phe Ile Glu Phe Leu Lys Asn-NH₂ [SEQ. ID. NO. 51]

The above-identified amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 37% to 47% Solvent B in Solvent A over 30 minutes) of the lyophilized peptide gave product peptide having an observed retention time of 17.9 minutes. Electrospray Mass Spectrometry (M): calculated 3179.6; found 3179.0.

### EXAMPLE 45

### Preparation of Peptide Having SEQ ID NO. 52

His Gly Glu Gly Thr Phe Thr Ser Asp Leu Ser Lys Gln Leu Glu Ala Glu Ala Val Arg Leu Phe Ile Glu Phe Leu Lys Asn-NH₂ [SEQ. ID. NO. 52]

The above-identified amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 37% to 47% Solvent B in Solvent A over 30 minutes) of the lyophilized peptide gave product peptide having an observed retention time of 14.3 minutes. Electrospray Mass Spectrometry (M): calculated 3179.6; found 3180.0.

### EXAMPLE 46

### Preparation of Peptide Having SEQ ID NO. 53

His Gly Glu Gly Thr Phe Thr Ser Asp Leu Ser Lys Gln Leu Glu Glu Ala Ala Val Arg Leu Phe Ile Glu Phe Leu Lys Asn-NH₂ [SEQ. ID. NO. 53]

The above-identified peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP- HPLC (gradient 37% to 47% Solvent B in Solvent A over 30 minutes) of the lyophilized peptide gave product peptide having an observed retention time of 13.7 minutes. Electrospray Mass Spectrometry (M): calculated 3179.6; found 3179.0.

### EXAMPLE 47

### Preparation of Peptide Having SEQ ID NO. 54

His Gly Glu Gly Thr Phe Thr Ser Asp Leu Ser Lys Gln Leu Glu Glu Glu Ala Ala Arg Leu Phe Ile Glu Phe Leu Lys Asn-NH₂ [SEQ. ID. NO. 54]

The above-identified amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 35% to 45% Solvent B in Solvent A over 30 minutes) of the lyophilized peptide gave product peptide having an observed retention time of 14.0 minutes. Electrospray Mass Spectrometry (M): calculated 3209.6; found 3212.8.

### EXAMPLE 48

### Preparation of Peptide Having SEQ ID NO. 55

His Gly Glu Gly Thr Phe Thr Ser Asp Leu Ser Lys Gln Leu Glu Glu Glu Ala Val Ala Leu Phe Ile Glu Phe Leu Lys Asn-NH₂ [SEQ. ID. NO. 55]

The above-identified amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 38% to 48% Solvent B in Solvent A over 30 minutes) of the lyophilized peptide gave product peptide having an observed retention time of 14.3 minutes. Electrospray Mass Spectrometry (M): calculated 3152.5; found 3153.5.

### EXAMPLE 49

### Preparation of Peptide Having SEQ ID NO. 56

His Gly Glu Gly Thr Phe Thr Ser Asp Leu Ser Lys Gln Leu Glu Glu Glu Ala Val Arg Ala Phe Ile Glu Phe Leu Lys Asn-NH₂ [SEQ. ID. NO. 56]

The above-identified amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 35% to 45% Solvent B in Solvent A over 30 minutes) of the lyophilized peptide gave product peptide having an observed retention time of 12.1 minutes. Electrospray Mass Spectrometry (M): calculated 3195.5; found 3197.7.

### EXAMPLE 50

### Preparation of Peptide Having SEQ ID NO. 57

His Gly Glu Gly Thr Phe Thr Ser Asp Leu Ser Lys Gln Leu Glu Glu Glu Ala Val Arg Leu Phe Ile Ala Phe Leu Lys Asn-NH₂ [SEQ. ID. NO. 57]

The above-identified amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 38% to 48% Solvent B in Solvent A over 30 minutes) of the lyophilized peptide gave product peptide having an observed retention time of 10.9 minutes. Electrospray Mass Spectrometry (M): calculated 3179.6; found 3180.5.

### EXAMPLE 51

### Preparation of Peptide Having SEQ ID NO. 58

His Gly Glu Gly Thr Phe Thr Ser Asp Leu Ser Lys Gln Leu Glu Glu Glu Ala Val Arg Leu Phe Ile Glu Ala Leu Lys Asn-NH₂ [SEQ. ID. NO. 58]

The above-identified amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 32% to 42% Solvent B in Solvent A over 30 minutes) of the lyophilized peptide gave product peptide having an observed retention time of 17.5 minutes. Electrospray Mass Spectrometry (M): calculated 3161.5; found 3163.0.

### EXAMPLE 52

### Preparation of Peptide Having SEQ ID NO. 59

His Gly Glu Gly Thr Phe Thr Ser Asp Leu Ser Lys Gln Leu Glu Glu Glu Ala Val Arg Leu Phe Ile Glu Phe Ala Lys Asn-NH₂ [SEQ. ID. NO. 59]

The above-identified amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 32% to 42% Solvent B in Solvent A over 30 minutes) of the lyophilized peptide gave product peptide having an observed retention time of 19.5 minutes. Electrospray Mass Spectrometry (M): calculated 3195.5; found 3199.

### EXAMPLE 53

### Preparation of Peptide Having SEQ ID NO. 60

His Gly Glu Gly Thr Phe Thr Ser Asp Leu Ser Lys Gln Leu Glu Glu Glu Ala Val Arg Leu Phe Ile Glu Phe Leu Ala Asn-NH₂ [SEQ. ID. NO. 60]

The above-identified amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 38% to 48% Solvent B in Solvent A over 30 minutes) of the lyophilized peptide gave product peptide having an observed retention time of 14.5 minutes. Electrospray Mass Spectrometry (M): calculated 3180.5; found 3183.7.

### EXAMPLE 54

### Preparation of Peptide Having SEQ ID NO. 61

His Gly Glu Gly Thr Phe Thr Ser Asp Leu Ser Lys Gln Leu Glu Glu Glu Ala Val Arg Leu Phe lie Glu Phe Leu Lys Ala-NH₂ [SEQ. ID. NO. 61]

The above-identified amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis were Solvent A (0.1% TFA in water) and Solvent B (0.1% TFA in ACN). Analytical RP-HPLC (gradient 34% to 44% Solvent B in Solvent A over 30 minutes) of the lyophilized peptide gave product peptide having an observed retention time of 22.8 minutes. Electrospray Mass Spectrometry (M): calculated 3194.6; found 3197.6.

### EXAMPLE 55

### Preparation of Peptide Having SEQ ID NO. 62

His Gly Glu Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala Pro Pro Pro-NH₂ [SEQ. ID. NO. 62]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4099.6.

### EXAMPLE 56

### Preparation of Peptide Having SEQ ID NO. 63

His Gly Glu Gly Thr Phe Thr Ser Asp Leu Ser Lys Gln Leu Glu Glu Glu Ala Val Arg Leu Phe Ile Glu Phe Leu Lys Asn Gly Gly Pro Ser Ser Gly Ala Pro Pro Pro-NH₂ [SEQ. ID. NO. 63]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4042.5.

### EXAMPLE 57

### Preparation of Peptide Having SEQ ID NO. 64

His Gly Glu Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala Pro Pro-NH₂ [SEQ. ID. NO. 64]

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4002.4

### EXAMPLE 58

### Preparation of Peptide Having SEQ ID NO. 65

His Gly Glu Gly Thr Phe Thr Ser Asp Leu Ser Lys Gln Leu Glu Glu Glu Ala Val Arg Leu Phe Ile Glu Phe Leu Lys Asn Gly Gly Pro Ser Ser Gly Ala Pro Pro-NH₂ [SEQ. ID. NO. 65]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3945.4.

### EXAMPLE 59

### Preparation of Peptide Having SEQ ID NO. 66

His Gly Glu Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala Pro-NH₂ [SEQ. ID. NO. 66]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3905.3.

### EXAMPLE 60

### Preparation of Peptide Having SEQ ID NO. 67

His Gly Glu Gly Thr Phe Thr Ser Asp Leu Ser Lys Gln Leu Glu Glu Glu Ala Val Arg Leu Phe Ile Glu Phe Leu Lys Asn Gly Gly Pro Ser Ser Gly Ala Pro-NH₂ [SEQ. ID. NO. 67]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3848.2.

### EXAMPLE 61

### Preparation of Peptide Having SEQ ID NO. 68

His Gly Glu Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala-NH₂ [SEQ. ID. NO. 68]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3808.2.

### EXAMPLE 62

### Preparation of Peptide Having SEQ ID NO. 69

His Gly Glu Gly Thr Phe Thr Ser Asp Leu Ser Lys Gln Leu Glu Glu Glu Ala Val Arg Leu Phe Ile Glu Phe Leu Lys Asn Gly Gly Pro Ser Ser Gly Ala-NH₂ [SEQ. ID. NO. 69]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3751.1.

### EXAMPLE 63

### Preparation of Peptide Having SEQ ID NO. 70

His Gly Glu Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly-NH₂ [SEQ. ID. NO. 70]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3737.1.

### EXAMPLE 64

### Preparation of Peptide Having SEQ ID NO. 71

His Gly Glu Gly Thr Phe Thr Ser Asp Leu Ser Lys Gln Leu Glu Glu Glu Ala Val Arg Leu Phe Ile Glu Phe Leu Lys Asn Gly Gly Pro Ser Ser Gly-NH₂ [SEQ. ID. NO. 71]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3680.1.

### EXAMPLE 65

### Preparation of Peptide Having SEQ ID NO. 72

His Gly Glu Gly Thr Phe Thr Ser Asp Leu Ser Lys Gln Met Glu Glu Glu Ala Val Arg Leu Phe Ile Glu Trp Leu Lys Asn Gly Gly Pro Ser Ser-NH₂ [SEQ. ID. NO. 72]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3680.1

### EXAMPLE 66

### Preparation of Peptide Having SEQ ID NO. 73

His Gly Glu Gly Thr Phe Thr Ser Asp Leu Ser Lys Gln Leu Glu Glu Glu Ala Val Arg Leu Phe Ile Glu Phe Leu Lys Asn Gly Gly Pro Ser Ser-NH₂ [SEQ. ID. NO. 73]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3623.0.

### EXAMPLE 67

### Preparation of Peptide Having SEQ ID NO. 74

His Gly Glu Gly Thr Phe Thr Ser Asp Leu Ser Lys Gln Met Glu Glu Glu Ala Val Arg Leu Phe Ile Glu Trp Leu Lys Asn Gly Gly Pro Ser-NH₂ [SEQ. ID. NO. 74]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3593.0

### EXAMPLE 68

### Preparation of Pentide Having SEQ ID NO. 75

His Gly Glu Gly Thr Phe Thr Ser Asp Leu Ser Lys Gln Leu Glu Glu Glu Ala Val Arg Leu Phe Ile Glu Phe Leu Lys Asn Gly Gly Pro Ser-NH₂ [SEQ. ID. NO. 75]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3535.9

### EXAMPLE 69

### Preparation of Peptide Having SEQ ID NO. 76

His Gly Glu Gly Thr Phe Thr Ser Asp Leu Ser Lys Gln Met Glu Glu Glu Ala Val Arg Leu Phe Ile Glu Trp Leu Lys Asn Gly Gly Pro-NH₂ [SEQ. ID. NO. 76]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3505.94.

### EXAMPLE 70

### Preparation of Peptide having SEQ ID NO. 77

His Gly Glu Gly Thr Phe Thr Ser Asp Leu Ser Lys Gin Leu Glu Glu Glu Ala Val Arg Leu Phe Ile Glu Phe Leu Lys Asn Gly Gly Pro-NH₂ [SEQ. ID. NO. 77]

The above-identified amidated peptide is assembled on 4-(2′-4′-dimethoxyphenyl)-Fmoc aminomethyl phenoxy acetamide norleucine MBHIA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFAin ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3448.8.

### EXAMPLE 71

### Preparation of Peptide Having SEQ ID NO. 78

His Gly Glu Gly Thr Phe Thr Ser Asp Leu Ser Lys Gin Leu Glu Glu Glu Ala Val Arg Leu Phe Ile Glu Phe Leu Lys Asn Gly Gly-NH₂ [SEQ. ID. NO. 78]

The above-identified peptide is assembled on 4-(2′-4′-dimethoxyphenyl)-Fmoc aminomethyl phenoxy acetamnide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1 % TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3351.7.

### EXAMPLE 72

### Preparation of Peptide Having SEQ ID NO. 79

His Gly Glu Gly Thr Phe Thr Ser Asp Leu Ser Lys Gln Met Glu Glu Glu Ala Val Arg Leu Phe Ile Glu Trp Leu Lys Asn Gly-NH₂ [SEQ. ID. NO. 79]

The above-identified peptide is assembled on 4-(2′-4′-dimethoxyphenyl)-Fmoc aminomethyl phenoxy acetamide norleucine MBHA resin (Novabiochem, 0.55 mmole/g) using Fmoc-protected arnino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3351.8.

### EXAMPLE 73

### Preparation of Peptide Having SEQ ID NO. 80

His Gly Glu Gly Thr Phe Thr Ser Asp Leu Ser Lys Gln Leu Glu Glu Glu Ala Val Arg Leu Phe Ile Glu Phe Leu Lys Asn Gly-NH₂ [SEQ. ID. NO. 80]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3294.7.

### EXAMPLE 74

### Preparation of Peptide Having SEQ ID NO. 81

His Gly Glu Gly Thr Phe Thr Ser Asp Leu Ser Lys Gln Met Glu Glu Glu Ala Val Arg Leu Phe Ile Glu Trp Leu Lys Asn Gly Gly tPro Ser Ser Gly Ala tPro tPro tPro-NH₂ [SEQ. ID. NO. 81]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Double couplings are required at residues 37,36 and 31. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4197.1.

### EXAMPLE 75

### Preparation of Peptide Having SEQ ID NO. 82

His Gly Glu Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala tPro tPro tPro-NH₂ [SEQ. ID. NO. 82]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Double couplings are required at residues 37, 36 and 31. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4179.1.

### EXAMPLE 76

### Preparation of Peptide Having SEQ ID NO. 83

His Gly Glu Gly Thr Phe Thr Ser Asp Leu Ser Lys Gln Met Glu Glu Glu Ala Val Arg Leu Phe Ile Glu Trp Leu Lys Asn Gly Gly NMeala Ser Ser Gly Ala Pro Pro-NH₂ [SEQ. ID. NO. 83]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Double couplings are required at residues 36 and 31. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3948.3.

### EXAMPLE 77

### Preparation of Peptide Having SEQ ID NO. 84

His Gly Glu Gly Thr Phe Thr Ser Asp Leu Ser Lys Gln Met Glu Glu Glu Ala Val Arg Leu Phe Ile Glu Trp Leu Lys Asn Gly Gly NMeala Ser Ser Gly Ala NMeala Nmeala-NH₂ [SEQ. ID. NO. 84]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Double couplings are required at residues 36 and 31. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3840.1.

### EXAMPLE 78

### Preparation of Peptide Having SEQ ID NO. 85

His Gly Glu Gly Thr Phe Thr Ser Asp Leu Ser Lys Gln Met Glu Glu Glu Ala Val Arg Leu Phe Ile Glu Trp Leu Lys Asn Gly Gly hPro Ser Ser Gly Ala hPro hPro-NH₂ [SEQ. ID. NO. 85]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Double couplings are required at residues 36 and 31. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4050.1.

### EXAMPLE 79

### Preparation of Peptide Having SEQ ID NO. 86

His Gly Glu Gly Thr Phe Thr Ser Asp Leu Ser Lys Gln Met Glu Glu Glu Ala Val Arg Leu Phe Ile Glu Trp Leu Lys Asn Gly Gly hPro Ser Ser Gly Ala hPro-NH₂ [SEQ. ID. NO. 86]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. A double coupling is required at residue 31. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3937.1

### EXAMPLE 80

### Preparation of Peptide Having SEQ ID NO. 87

Arg Gly Glu Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala-NH2 [SEQ. ID. NO. 87]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3827.2.

### EXAMPLE 81

### Preparation of Peptide Having SEQ ID NO. 88

His Gly Asp Gly Thr Phe Thr Ser Asp Leu Ser Lys Gln Met Glu Glu Glu Ala Val Arg Leu Phe Ile Glu Trp Leu Lys Asn Gly Gly-NH₂ [SEQ. ID. NO. 88]

The above-identified amidated peptide is assembled on 4-(2′-4′-dimethoxyphenyl)-Fmoc aminomethyl phenoxy acetamnide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3394.8.

### EXAMPLE 82

### Preparation of Peptide Having SEQ ID NO. 89

His Gly Glu Gly Thr Naphthylala Thr Ser Asp Leu Ser Lys Gln Leu Glu Gin Glu Ala Val Arg Leu Phe Ile Glu Phe Leu Lys Asn-NH₂ [SEQ. ID. NO. 89]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3289.5.

### EXAMPLE 83

### Preparation of Peptide having SEQ ID NO. 90

His Gly Glu Gly Thr Phe Ser Ser Asp Leu Ser Lys Gln Met Glu Glu Glu Ala Val Arg Leu Phe Ile Glu Trp Leu Lys Asn-NH₂ [SEQ. ID. NO. 90]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3280.7.

### EXAMPLE 84

### Preparation of Peptide having SEQ ID NO. 91

His Gly Glu Gly Thr Phe Ser Thr Asp Leu Ser Lys Gln Met Glu Glu Glu Ala Val Arg Leu Phe Ile Glu Trp Leu Lys Asn-NH₂ [SEQ. ID. NO. 91]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3294.7.

### EXAMPLE 85

### Preparation of Peptide Having SEQ ID NO. 92

His Gly Glu Gly Thr Phe Thr Ser Glu Leu Ser Lys Gln Met Ala Glu Glu Ala Val Arg Leu Phe Ile Glu Trp Leu Lys Asn-NH₂ [SEQ. ID. NO. 92]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3250.7.

### EXAMPLE 86

### Preparation of Peptide having SEQ ID NO. 93

His Gly Glu Gly Thr Phe Thr Ser Asp pentylgly Ser Lys Gln Leu Glu Glu Glu Ala Val Arg Leu Phe Ile Glu Phe Leu Lys Asn-NH₂ [SEQ. ID. NO. 93]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3253.5.

### EXAMPLE 87

### Preparation of Peptide Having SEQ ID NO. 94

His Gly Glu Gly Thr Phe Thr Ser Asp Leu Ser Lys Gln Leu Glu Glu Glu Ala Val Arg Leu Naphthylala Ile Glu Phe Leu Lys Asn-NH₂ [SEQ. ID. NO. 94]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3289.5.

### EXAMPLE 88

### Preparation of Peptide Having SEQ ID NO. 95

His Gly Glu Gly Thr Phe Thr Ser Asp Leu Ser Lys Gln Met Glu Glu Glu Ala Val Arg Leu Phe tButylgly Glu Trp Leu Lys Asn-NH₂ [SEQ. ID. NO. 95]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3183.4.

### EXAMPLE 89

### Preparation of Peptide Having SEQ ID NO. 96

His Gly Glu Gly Thr Phe Thr Ser Asp Leu Ser Lys Gln Leu Glu Glu Glu Ala Val Arg Leu Phe Ile Asp Phe Leu Lys Asn-NH₂ [SEQ. ID. NO. 96]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3237.6.

### EXAMPLE 90

### Preparation of Peptide Having SEQ ID NO. 97

His Gly Glu Gly Thr Phe Thr Ser Asp Ala Ser Lys Gln Leu Glu Glu Glu Ala Val Arg Leu Phe Ile Glu Phe Leu Lys Asn Gly Gly Pro Ser Ser-NH₂ [SEQ. ID. NO. 97]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3637.9.

### EXAMPLE 91

### Preparation of Peptide Having SEQ ID NO. 98

His Gly Glu Gly Thr Phe Thr Ser Asp Ala Ser Lys Gln Met Glu Glu Glu Ala Val Arg Leu Phe Ile Glu Trp Leu Lys Asn Gly-NH₂ [SEQ. ID. NO. 98]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3309.7.

### EXAMPLE 92

### Preparation of Peptide Having SEQ ID NO. 99

His Gly Glu Gly Thr Phe Thr Ser Asp Ala Ser Lys Gln Met Glu Glu Glu Ala Val Arg Leu Phe Ile Glu Trp Leu Lys Asn Gly Gly hPro Ser Ser Gly Ala hPro hPro-NH₂ [SEQ. ID. NO. 99]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 32. Double couplings are required at residues 36 and 31. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3711.1.

### EXAMPLE 93

### Preparation of C-terminal Carboxylic Acid Peptides Corresponding to the Above C-terminal Amide Sequences for SEQ ID NOS. 7, 40-61, 68-75, 78-80 and 87-98

Peptides having the sequences of SEQ ID NOS. 7, 40-61, 68-75, 78-80 and 87-98 are assembled on the so called Wang resin (p-alkoxybenzylalacohol resin (Bachem, 0.54 mmole/g)) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry provides an experimentally determined (M).

### EXAMPLE 94

### Preparation of C-terminal carboxylic Acid Peptides Corresponding to the Above C-terminal Amide Sequences for SEQ ID NOS. 62-67, 76, 77, 81-86 and 99

Peptides having the sequences of SEQ ID NOS. 62-67, 76, 77, 81-86 and 99 are assembled on the 2-chlorotritylchloride resin (200-400 mesh), 2% DVB (Novabiochem, 0.4-1.0 mmole/g)) using Fmoc-protected amino acids (Applied Biosystems, Inc.), cleaved from the resin, deprotected and purified in a similar way to Example 32. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry provides an experimentally determined (M).

### EXAMPLE 95

### Preparation of Peptide Having SEQ ID NO. 100

Ala Gly Glu Gly Thr Phe Thr Ser Asp Leu Ser Lys Gln Leu Glu Glu Glu Ala Val Arg Leu Phe Ile Glu Phe Leu Lys Asn-NH₂ [SEQ. ID. NO. 100]

The above amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.). In general, single-coupling cycles were used throughout the synthesis and Fast Moc (HBTU activation) chemistry was employed. Deprotection (Fmoc group removal) of the growing peptide chain was achieved using piperidine. Final deprotection of the completed peptide resin was achieved using a mixture of triethylsilane (0.2 mL), ethanedithiol (0.2 mL), anisole (0.2 mL), water (0.2 mL) and trifluoroacetic acid (15 mL) according to standard methods (Introduction to Cleavage Techniques, Applied Biosystems, Inc.) The peptide was precipitated in ether/water (50 mL) and centrifuged. The precipitate was reconstituted in glacial acetic acid and lyophilized. The lyophilized peptide was dissolved in water). Crude purity was about 75%.

Used in purification steps and analysis were Solvent A (0.1% TFA in water) and Solvent B (0.1% TFA in ACN).

The solution containing peptide was applied to a preparative C-18 column and purified (10% to 40% Solvent B in Solvent A over 40 minutes). Purity of fractions was determined isocratically using a C-18 analytical column. Pure fractions were pooled furnishing the above-identified peptide. Analytical RP-HPLC (gradient 30% to 60% Solvent B in Solvent A over 30 minutes) of the lyophilized peptide gave product peptide having an observed retention time of 19.2 minutes. Electrospray Mass Spectrometry (M): calculated 3171.6; found 3172.

### EXAMPLE 96

### Preparation of Peptide Having SEQ ID NO. 101

His Gly Ala Gly Thr Phe Thr Ser Asp Leu Ser Lys Gln Leu Glu Glu Glu Ala Val Arg Leu Phe Ile Glu Phe Leu Lys Asn-NH₂ [SEQ. ID. NO. 101]

The above amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis were Solvent A (0.1% TFA in water) and Solvent B (0.1% TFA in ACN). Analytical RP-HPLC (gradient 36% to 46% Solvent B in Solvent A over 30 minutes) of the lyophilized peptide gave product peptide having an observed retention time of 14.9 minutes. Electrospray Mass Spectrometry (M): calculated 3179.6; found 3180.

### EXAMPLE 97

### Preparation of Peptide Having SEQ ID NO. 102

His Gly Glu Ala Thr Phe Thr Ser Asp Leu Ser Lys Gln Leu Glu Glu Glu Ala Val Arg Leu Phe Ile Glu Phe Leu Lys Asn-NH₂ [SEQ. ID. NO. 102]

The above amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis were Solvent A (0.1% TFA in water) and Solvent B (0.1% TFA in ACN). Analytical RP-HPLC (gradient 37% to 47% Solvent B in Solvent A over 30 minutes) of the lyophilized peptide gave product peptide having an observed retention time of 12.2 minutes. Electrospray Mass Spectrometry (M): calculated 3251.6; found 3253.3.

### EXAMPLE 98

### Preparation of Peptide Having SEQ ID NO. 103

His Gly Glu Gly Thr Phe Thr Ser Ala Leu Ser Lys Gln Leu Glu Glu Glu Ala Val Arg Leu Phe Ile Glu Phe Leu Lys Asn-NH₂ [SEQ. ID. NO. 103]

The above amidated peptide was assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis were Solvent A (0.1% TFA in water) and Solvent B (0.1% TFA in ACN). Analytical RP-HPLC (gradient 35% to 45% Solvent B in Solvent A over 30 minutes) of the lyophilized peptide gave product peptide having an observed retention time of 16.3 minutes. Electrospray Mass Spectrometry (M): calculated 3193.6; found 3197.

### EXAMPLE 99

### Preparation of Peptide Having SEQ ID NO. 104

Ala Gly Glu Gly Thr Phe Thr Ser Asp Leu Ser Lys Gln Met Glu Glu Glu Ala Val Arg Leu Phe Ile Glu Trp Leu Lys Asn-NH₂ [SEQ. ID. NO. 104]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3228.6.

### EXAMPLE 100

### Preparation of Peptide Having SEQ ID NO. 105

His Gly Ala Gly Thr Phe Thr Ser Asp Leu Ser Lys Gln Met Glu Glu Glu Ala Val Arg Leu Phe Ile Glu Trp Leu Lys Asn-NH₂ [SEQ. ID. NO. 105]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3234.7.

### EXAMPLE 101

### Preparation of Peptide Having SEQ ID NO. 106

His Gly Glu Ala Thr Phe Thr Ser Asp Leu Ser Lys Gln Met Glu Glu Glu Ala Val Arg Leu Phe Ile Glu Trp Leu Lys Asn-NH₂ [SEQ. ID. NO. 106]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3308.7.

### EXAMPLE 102

### Preparation of Peptide Having SEQ ID NO. 107

His Gly Glu Gly Thr Phe Thr Ser Ala Leu Ser Lys Gln Met Glu Glu Glu Ala Val Arg Leu Phe Ile Glu Trp Leu Lys Asn-NH₂ [SEQ. ID. NO. 107]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3250.7

### EXAMPLE 103

### Preparation of Peptide Having SEQ ID NO. 108

His Gly Glu Gly Thr Phe Thr Ser Asp Ala Ser Lys Gln Met Glu Glu Glu Ala Val Arg Leu Phe Ile Glu Trp Leu Lys Asn-NH₂ [SEQ. ID. NO. 108]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3252.6.

### EXAMPLE 104

### Preparation of Peptide Having SEQ ID NO. 109

Ala Ala Glu Gly Thr Phe Thr Ser Asp Leu Ser Lys Gln Met Glu Glu Glu Ala Val Arg Leu Phe Ile Glu Trp Leu Lys Asn-NH₂ [SEQ. ID. NO. 109]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3200.6.

### EXAMPLE 105

### Preparation of Peptide Having SEQ ID NO. 110

Ala Ala Glu Gly Thr Phe Thr Ser Asp Leu Ser Lys Gln Leu Glu Glu Glu Ala Val Arg Leu Phe Ile Glu Phe Leu Lys Asn-NH₂ [SEQ. ID. NO. 110]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3143.5.

### EXAMPLE 106

### Preparation of Peptide Having SEQ ID NO. 111

Ala Gly Asp Gly Thr Phe Thr Ser Asp Leu Ser Lys Gln Met Glu Glu Glu Ala Val Arg Leu Phe Ile Glu Trp Leu Lys Asn-NH₂ [SEQ. ID. NO. 111]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3214.6.

### EXAMPLE 107

### Preparation of Peptide Having SEQ ID NO. 112

Ala Gly Asp Gly Thr Phe Thr Ser Asp Leu Ser Lys Gln Leu Glu Glu Glu Ala Val Arg Leu Phe Ile Glu Phe Leu Lys Asn-NH₂ [SEQ. ID. NO. 112]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3157.5.

### EXAMPLE 108

### Preparation of Peptide Having SEQ ID NO. 113

Ala Gly Asp Gly Ala Phe Thr Ser Asp Leu Ser Lys Gln Met Glu Glu Glu Ala Val Arg Leu Phe Ile Glu Trp Leu Lys Asn-NH₂ [SEQ. ID. NO. 113]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3184.6.

### EXAMPLE 109

### Preparation of Peptide Having SEQ ID NO. 114

Ala Gly Asp Gly Ala Phe Thr Ser Asp Leu Ser Lys Gln Leu Glu Glu Glu Ala Val Arg Leu Phe Ile Glu Phe Leu Lys Asn-NH₂ [SEQ. ID. NO. 114]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3127.5.

### EXAMPLE 110

### Preparation of Peptide Having SEQ ID NO. 115

Ala Gly Asp Gly Thr NaphthylAla Thr Ser Asp Leu Ser Lys Gln Met Glu Glu Glu Ala Val Arg Leu Phe Ile Glu Trp Leu Lys Asn-NH₂ [SEQ. ID. NO. 115]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3266.4.

### EXAMPLE 111

### Preparation of Peptide Having SEQ ID NO. 116

Ala Gly Asp Gly Thr Naphthylala Thr Ser Asp Leu Ser Lys Gln Leu Glu Glu Glu Ala Val Arg Leu Phe Ile Glu Phe Leu Lys Asn-NH₂ [SEQ. ID. NO. 116]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3209.4.

### EXAMPLE 112

### Preparation of Peptide Having SEQ ID NO. 117

Ala Gly Asp Gly Thr Phe Ser Ser Asp Leu Ser Lys Gln Met Glu Glu Glu Ala Val Arg Leu Phe Ile Glu Trp Leu Lys Asn-NH₂ [SEQ. ID. NO. 117]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3200.6.

### EXAMPLE 113

### Preparation of Peptide Having SEQ ID NO. 118

Ala Gly Asp Gly Thr Phe Ser Ser Asp Leu Ser Lys Gln Leu Glu Glu Glu Ala Val Arg Leu Phe Ile Glu Phe Leu Lys Asn-NH₂ [SEQ. ID. NO. 118]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3143.5.

### EXAMPLE 114

### Preparation of Peptide Having SEQ ID NO. 119

Ala Gly Asp Gly Thr Phe Thr Ala Asp Leu Ser Lys Gln Met Glu Glu Glu Ala Val Arg Leu Phe Ile Glu Trp Leu Lys Asn-NH₂ [SEQ. ID. NO. 119]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3198.6.

### EXAMPLE 115

### Preparation of Peptide Having SEQ ID NO. 120

Ala Gly Asp Gly Thr Phe Thr Ala Asp Leu Ser Lys Gln Leu Glu Glu Glu Ala Val Arg Leu Phe Ile Glu Phe Leu Lys Asn-NH₂ [SEQ. ID. NO. 120]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3141.5.

### EXAMPLE 116

### Preparation of Peptide Having SEQ ID NO. 121

Ala Gly Asp Gly Thr Phe Thr Ser Ala Leu Ser Lys Gln Met Glu Glu Glu Ala Val Arg Leu Phe Ile Glu Trp Leu Lys Asn-NH₂ [SEQ. ID. NO. 121]

The above-identified peptide is assembled on 4-(2′-4′-dimethoxyphenyl)-Fmoc amino methyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3170.6.

### EXAMPLE 117

### Preparation of Peptide Having SEQ ID NO. 122

Ala Gly Asp Gly Thr Phe Thr Ser Ala Leu Ser Lys Gln Leu Glu Glu Glu Ala Val Arg Leu Phe Ile Glu Phe Leu Lys Asn-NH₂ [SEQ. ID. NO. 122]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3113.5.

### EXAMPLE 118

### Preparation of Peptide Having SEQ ID NO. 123

Ala Gly Asp Gly Thr Phe Thr Ser Glu Leu Ser Lys Gln Met Glu Glu Glu Ala Val Arg Leu Phe Ile Glu Trp Leu Lys Asn-NH₂ [SEQ. ID. NO. 123]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3228.6.

### EXAMPLE 119

### Preparation of Peptide Having SEQ ID NO. 124

Ala Gly Asp Gly Thr Phe Thr Ser Glu Leu Ser Lys Gln Leu Glu Glu Glu Ala Val Arg Leu Phe Ile Glu Phe Leu Lys Asn-NH₂ [SEQ. ID. NO. 124]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3171.6.

### EXAMPLE 120

### Preparation of Peptide Having SEQ ID NO. 125

Ala Gly Asp Gly Thr Phe Thr Ser Asp Ala Ser Lys Gln Met Glu Glu Glu Ala Val Arg Leu Phe Ile Glu Trp Leu Lys Asn-NH₂ [SEQ. ID. NO. 125]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3172.5.

### EXAMPLE 121

### Preparation of Peptide Having SEQ ID NO. 126

Ala Gly Asp Gly Thr Phe Thr Ser Asp Ala Ser Lys Gln Leu Glu Glu Glu Ala Val Arg Leu Phe Ile Glu Phe Leu Lys Asn-NH₂ [SEQ. ID. NO. 126]

The above-identified amidated peptiden is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3115.4.

### EXAMPLE 122

### Preparation of Peptide Having SEQ ID NO. 127

Ala Gly Asp Gly Thr Phe Thr Ser Asp Pentylgly Ser Lys Gln Met Glu Glu Glu Ala Val Arg Leu Phe Ile Glu Trp Leu Lys Asn-NH₂ [SEQ. ID. NO. 127]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3230.4.

### EXAMPLE 123

### Preparation of Peptide Having SEO ID NO. 128

Ala Gly Asp Gly Thr Phe Thr Ser Asp Pentylgly Ser Lys Gln Leu Glu Glu Glu Ala Val Arg Leu Phe Ile Glu Phe Leu Lys Asn-NH₂ [SEQ. ID. NO. 128]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3198.6.

### EXAMPLE 124

### Preparation of Peptide Having SEQ ID NO. 129

Ala Gly Asp Gly Thr Phe Thr Ser Asp Leu Ala Lys Gln Met Glu Glu Glu Ala Val Arg Leu Phe Ile Glu Trp Leu Lys Asn-NH₂ [SEQ. ID. NO. 129]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3141.5.

### EXAMPLE 125

### Preparation of Peptide Having SEQ ID NO. 130

Ala Gly Asp Gly Thr Phe Thr Ser Asp Leu Ala Lys Gln Leu Glu Glu Glu Ala Val Arg Leu Phe Ile Glu Phe Leu Lys Asn-NH₂ [SEQ. ID. NO. 130]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3157.5.

### EXAMPLE 126

### Preparation of Peptide Having SEQ ID NO. 131

Ala Gly Asp Gly Thr Phe Thr Ser Asp Leu Ser Ala Gln Met Glu Glu Glu Ala Val Arg Leu Phe Ile Glu Trp Leu Lys Asn-NH₂ [SEQ. ID. NO. 131]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3100.4.

### EXAMPLE 127

### Preparation of Peptide Having SEQ ID NO. 132

Ala Gly Asp Gly Thr Phe Thr Ser Asp Leu Ser Ala Gln Leu Glu Glu Glu Ala Val Arg Leu Phe Ile Glu Phe Leu Lys Asn-NH₂ [SEQ. ID. NO. 132]

The above-identified amidated peptide is assembled on 4-(2′-4′-dimethoxyphenyl)-Fmoc arninomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3157.6.

### EXAMPLE 128

### Preparation of Peptide Having SEQ ID NO. 133

Ala Gly Asp Gly Thr Phe Thr Ser Asp Leu Ser Lys Ala Met Glu Glu Glu Ala Val Arg Leu Phe Ile Glu Trp Leu Lys Asn-NH₂ [SEQ. ID. NO. 133]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3100.5.

### EXAMPLE 129

### Preparation of Peptide Having SEQ ID NO. 134

Ala Gly Asp Gly Thr Phe Thr Ser Asp Leu Ser Lys Ala Leu Glu Glu Glu Ala Val Arg Leu Phe Ile Glu Phe Leu Lys Asn-NH₂ [SEQ. ID. NO. 134]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3100.5.

### EXAMPLE 130

### Preparation of Peptide Having SEQ ID NO. 135

Ala Gly Asp Gly Thr Phe Thr Ser Asp Leu Ser Lys Gln Ala Glu Glu Glu Ala Val Arg Leu Phe Ile Glu Trp Leu Lys Asn-NH₂ [SEQ. ID. NO. 135]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3154.5.

### EXAMPLE 131

### Preparation of Peptide Having SEQ ID NO. 136

Ala Gly Asp Gly Thr Phe Thr Ser Asp Leu Ser Lys Gln Ala Glu Glu Glu Ala Val Arg Leu Phe Ile Glu Phe Leu Lys Asn-NH₂ [SEQ. ID. NO. 136]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3115.5.

### EXAMPLE 132

### Preparation of Peptide Having SEQ ID NO. 137

Ala Gly Asp Gly Thr Phe Thr Ser Asp Leu Ser Lys Gln Pentylgly Glu Glu Glu Ala Val Arg Leu Phe Ile Glu Trp Leu Lys Asn-NH₂ [SEQ. ID. NO. 137]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3212.4.

### EXAMPLE 133

### Preparation of Peptide Having SEQ ID NO. 138

Ala Gly Asp Gly Thr Phe Thr Ser Asp Leu Ser Lys Gln Pentylgly Glu Glu Glu Ala Val Arg Leu Phe Ile Glu Phe Leu Lys Asn-NH₂ [SEQ. ID. NO. 138]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3173.4.

### EXAMPLE 134

### Preparation of Peptide Having SEQ ID NO. 139

Ala Gly Asp Gly Thr Phe Thr Ser Asp Leu Ser Lys Gln Met Ala Glu Glu Ala Val Arg Leu Phe Ile Glu Trp Leu Lys Asn-NH₂ [SEQ. ID. NO. 139]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3156.6.

### EXAMPLE 135

### Preparation of Peptide Having SEO ID NO. 140

Ala Gly Asp Gly Thr Phe Thr Ser Asp Leu Ser Lys Gln Leu Ala Glu Glu Ala Val Arg Leu Phe Ile Glu Phe Leu Lys Asn-NH₂ [SEQ. ID. NO. 140]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-UPLC (gradient 30% to 60% Solvent B in Solvent A over 30 minutes) of the lyophilized peptide is then carried out to determine the retention time of the product peptide. Electrospray Mass Spectrometry (M): calculated 3099.5.

### EXAMPLE 136

### Preparation of Peptide Having SEQ ID NO. 141

Ala Gly Asp Gly Thr Phe Thr Ser Asp Leu Ser Lys Gln Met Glu Ala Glu Ala Val Arg Leu Phe Ile Glu Trp Leu Lys Asn-NH₂ [SEQ. ID. NO. 141]

The above-identified amidated peptide is assembled on 4-(2′-4′-dimethoxyphenyl)-Fmoc arninomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3156.6.

### EXAMPLE 137

### Preparation of Peptide Having SEQ ID NO. 142

Ala Gly Asp Gly Thr Phe Thr Ser Asp Leu Ser Lys Gln Leu Glu Ala Glu Ala Val Arg Leu Phe Ile Glu Phe Leu Lys Asn-NH₂ [SEQ. ID. NO. 142]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3099.5.

### EXAMPLE 138

### Preparation of Peptide Having SEQ ID NO. 143

Ala Gly Asp Gly Thr Phe Thr Ser Asp Leu Ser Lys Gln Met Glu Glu Ala Ala Val Arg Leu Phe Ile Glu Trp Leu Lys Asn-NH₂ [SEQ. ID. NO. 143]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3156.6.

### EXAMPLE 139

### Preparation of Peptide Having SEQ ID NO. 144

Ala Gly Asp Gly Thr Phe Thr Ser Asp Leu Ser Lys Gln Leu Glu Glu Ala Ala Val Arg Leu Phe Ile Glu Phe Leu Lys Asn-NH₂ [SEQ. ID. NO. 144]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3099.5.

### EXAMPLE 140

### Preparation of Peptide Having SEQ ID NO. 145

Ala Gly Asp Gly Thr Phe Thr Ser Asp Leu Ser Lys Gln Met Glu Glu Glu Ala Ala Arg Leu Phe Ile Glu Trp Leu Lys Asn-NH₂ [SEQ. ID. NO. 145]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3186.6.

### EXAMPLE 141

### Preparation of Peptide Having SEQ ID NO. 146

Ala Gly Asp Gly Thr Phe Thr Ser Asp Leu Ser Lys Gln Leu Glu Glu Glu Ala Ala Arg Leu Phe Ile Glu Phe Leu Lys Asn-NH₂ [SEQ. ID. NO. 146]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3129.5.

### EXAMPLE 142

### Preparation of Peptide Having SEQ ID NO. 147

Ala Gly Asp Gly Thr Phe Thr Ser Asp Leu Ser Lys Gln Met Glu Glu Glu Ala Val Ala Leu Phe Ile Glu Trp Leu Lys Asn-NH₂ [SEQ. ID. NO. 147]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3129.5.

### EXAMPLE 143

### Preparation of Peptide Having SEQ ID NO. 148

Ala Gly Asp Gly Thr Phe Thr Ser Asp Leu Ser Lys Gln Leu Glu Glu Glu Ala Val Ala Leu Phe Ile Glu Phe Leu Lys Asn-NH₂ [SEQ. ID. NO. 148]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3072.4.

### EXAMPLE 144

### Preparation of Peptide Having SEQ ID NO. 149

Ala Gly Asp Gly Thr Phe Thr Ser Asp Leu Ser Lys Gln Met Glu Glu Glu Ala Val Arg Ala Phe Ile Glu Trp Leu Lys Asn-NH₂ [SEQ. ID. NO. 149]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3172.5.

### EXAMPLE 145

### Preparation of Peptide Having SEQ ID NO. 150

Ala Gly Asp Gly Thr Phe Thr Ser Asp Leu Ser Lys Gln Leu Glu Glu Glu Ala Val Arg Ala Phe Ile Glu Phe Leu Lys Asn-NH₂ [SEQ. ID. NO. 150]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3115.5.

### EXAMPLE 146

### Preparation of Peptide Having SEQ ID NO. 151

Ala Gly Asp Gly Thr Phe Thr Ser Asp Leu Ser Lys Gln Met Glu Glu Glu Ala Val Arg Leu Naphthylala Ile Glu Trp Leu Lys Asn-NH₂ [SEQ. ID. NO. 151]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3266.4.

### EXAMPLE 147

### Preparation of Peptide Having SEQ ID NO. 152

Ala Gly Asp Gly Thr Phe Thr Ser Asp Leu Ser Lys Gln Leu Glu Glu Glu Ala Val Arg Leu Naphthylala Ile Glu Phe Leu Lys Asn-NH2 [SEQ. ID. NO. 152]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3209.4.

### EXAMPLE 148

### Preparation of Peptide Having SEQ ID NO. 153

Ala Gly Asp Gly Thr Phe Thr Ser Asp Leu Ser Lys Gln Met Glu Glu Glu Ala Val Arg Leu Phe Val Glu Trp Leu Lys Asn-NH₂ [SEQ. ID. NO. 153]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3200.6.

### EXAMPLE 149

### Preparation of Peptide Having SEQ ID NO. 154

Ala Gly Asp Gly Thr Phe Thr Ser Asp Leu Ser Lys Gin Leu Glu Glu Glu Ala Val Arg Leu Phe Val Glu Phe Leu Lys Asn-NH₂ [SEQ. ID. NO. 154]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3143.5.

### EXAMPLE 150

### Preparation of Peptide Having SEQ ID NO. 155

Ala Gly Asp Gly Thr Phe Thr Ser Asp Leu Ser Lys Gln Met Glu Glu Glu Ala Val Arg Leu Phe tButylgly Glu Trp Leu Lys Asn-NH₂ [SEQ. ID. NO. 155]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3216.5.

### EXAMPLE 151

### Preparation of Peptide Having SEQ ID NO. 156

Ala Gly Asp Gly Thr Phe Thr Ser Asp Leu Ser Lys Gln Leu Glu Glu Glu Ala Val Arg Leu Phe tButylgly Glu Phe Leu Lys Asn-NH₂ [SEQ. ID. NO. 156]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3159.4.

### EXAMPLE 152

### Preparation of Peptide Having SEQ ID NO. 157

Ala Gly Asp Gly Thr Phe Thr Ser Asp Leu Ser Lys Gln Met Glu Glu Glu Ala Val Arg Leu Phe Ile Asp Trp Leu Lys Asn-NH₂ [SEQ. ID. NO. 157]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3200.6.

### EXAMPLE 153

### Preparation of Peptide Having SEQ ID NO. 158

Ala Gly Asp Gly Thr Phe Thr Ser Asp Leu Ser Lys Gln Leu Glu Glu Glu Ala Val Arg Leu Phe Ile Asp Phe Leu Lys Asn-NH₂ [SEQ. ID. NO. 158]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3143.5.

### EXAMPLE 154

### Preparation of Peptide Having SEQ ID NO. 159

Ala Gly Asp Gly Thr Phe Thr Ser Asp Leu Ser Lys Gln Met Glu Glu Glu Ala Val Arg Leu Phe Ile Glu Ala Leu Lys Asn-NH₂ [SEQ. ID. NO. 159]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3099.5.

### EXAMPLE 155

### Preparation of Peptide Having SEQ ID NO. 160

Ala Gly Asp Gly Thr Phe Thr Ser Asp Leu Ser Lys Gln Leu Glu Glu Glu Ala Val Arg Leu Phe Ile Glu Ala Leu Lys Asn-NH₂ [SEQ. ID. NO. 160]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3081.4.

### EXAMPLE 156

### Preparation of Peptide Having SEQ ID NO. 161

Ala Gly Asp Gly Thr Phe Thr Ser Asp Leu Ser Lys Gln Met Glu Glu Glu Ala Val Arg Leu Phe Ile Glu Trp Ala Lys Asn-NH₂ [SEQ. ID. NO. 161]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3172.5.

### EXAMPLE 157

### Preparation of Peptide Having SEQ ID NO. 162

Ala Gly Asp Gly Thr Phe Thr Ser Asp Leu Ser Lys Gln Leu Glu Glu Glu Ala Val Arg Leu Phe Ile Glu Phe Ala Lys Asn-NH₂ [SEQ. ID. NO. 162]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3115.5.

### EXAMPLE 158

### Preparation of Peptide Having SEQ ID NO. 163

Ala Gly Asp Gly Thr Phe Thr Ser Asp Leu Ser Lys Gln Met Glu Glu Glu Ala Val Arg Leu Phe Ile Glu Trp Leu Ala Asn-NH₂ [SEQ. ID. NO. 163]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3157.5.

### EXAMPLE 159

### Preparation of Peptide Having SEQ ID NO. 164

Ala Gly Asp Gly Thr Phe Thr Ser Asp Leu Ser Lys Gln Leu Glu Glu Glu Ala Val Arg Leu Phe Ile Glu Phe Leu Ala Asn-NH₂ [SEQ. ID. NO. 164]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3100.4.

### EXAMPLE 160

### Preparation of Peptide Having SEQ ID NO. 165

Ala Gly Asp Gly Thr Phe Thr Ser Asp Leu Ser Lys Gln Met Glu Glu Glu Ala Val Arg Leu Phe Ile Glu Trp Leu Lys Ala-NH₂ [SEQ. ID. NO. 165]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3171.6.

### EXAMPLE 161

### Preparation of Peptide Having SEQ ID NO. 166

Ala Gly Asp Gly Thr Phe Thr Ser Asp Leu Ser Lys Gln Leu Glu Glu Glu Ala Val Arg Leu Phe Ile Glu Phe Leu Lys Ala-NH₂ [SEQ. ID. NO. 166]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3114.5.

### EXAMPLE 162

### Preparation of Peptide Having SEQ ID NO. 167

Ala Gly Glu Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala Pro Pro Pro-NH₂ [SEQ. ID. NO. 167]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4033.5.

### EXAMPLE 163

### Preparation of Peptide Having SEQ ID NO. 168

His Gly Ala Gly Thr Phe Thr Ser Asp Leu Ser Lys Gln Leu Glu Glu Glu Ala Val Arg Leu Phe Ile Glu Phe Leu Lys Asn Gly Gly Pro Ser Ser Gly Ala Pro Pro Pro-NH₂ [SEQ. ID. NO. 168]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3984.4.

### EXAMPLE 164

### Preparation of Peptide Having SEQ ID NO. 169

His Gly Glu Ala Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala Pro Pro-NH₂ [SEQ. ID. NO. 169]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4016.5.

### EXAMPLE 165

### Preparation of Peptide Having SEQ ID NO. 170

His Gly Glu Gly Thr Phe Thr Ser Ala Leu Ser Lys Gln Met Glu Glu Glu Ala Val Arg Leu Phe Ile Glu Trp Leu Lys Asn Gly Gly Pro Ser Ser Gly Ala Pro-NH₂ [SEQ. ID. NO. 170]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3861.3.

### EXAMPLE 166

### Preparation of Peptide Having SEQ ID NO. 171

Ala Gly Glu Gly Thr Phe Thr Ser Asp Ala Ser Lys Gln Leu Glu Glu Glu Ala Val Arg Leu Phe Ile Glu Phe Leu Lys Asn Gly Gly Pro Ser Ser Gly Ala Pro-NH₂ [SEQ. ID. NO. 171]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3746.1.

### EXAMPLE 167

### Preparation of Peptide Having SEQ ID NO. 172

Ala Gly Glu Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala-NH₂ [SEQ. ID. NO. 172]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3742.1.

### EXAMPLE 168

### Preparation of Peptide Having SEQ ID NO. 173

His Gly Ala Gly Thr Phe Thr Ser Asp Leu Ser Lys Gln Leu Glu Glu Glu Ala Val Arg Leu Phe Ile Glu Phe Leu Lys Asn Gly Gly Pro Ser Ser Gly Ala-NH₂ [SEQ. ID. NO. 173]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3693.1.

### EXAMPLE 169

### Preparation of Peptide Having SEQ ID NO. 174

His Gly Glu Ala Thr Phe Thr Ser Asp Leu Ser Lys Gln Met Glu Glu Glu Ala Val Arg Leu Phe Ile Glu Trp Leu Lys Asn Gly Gly Pro Ser Ser Gly-NH₂ [SEQ. ID. NO. 174]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3751.2.

### EXAMPLE 170

### Preparation of Peptide Having SEQ ID NO. 175

His Gly Glu Gly Thr Phe Thr Ser Ala Leu Ser Lys Gln Met Glu Glu Glu Ala Val Arg Leu Phe Ile Glu Trp Leu Lys Asn Gly Gly Pro Ser Ser-NH₂ [SEQ. ID. NO. 175]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3634.1.

### EXAMPLE 171

### Preparation of Peptide Having SEQ ID NO. 176

Ala Gly Glu Gly Thr Phe Thr Ser Asp Leu Ser Lys Gln Met Glu Glu Glu Ala Val Arg Leu Phe Ile Glu Trp Leu Lys Asn Gly Gly Pro Ser-NH₂ [SEQ. ID. NO. 176]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3526.9.

### EXAMPLE 172

### Preparation of Peptide Having SEQ ID NO. 177

His Gly Ala Gly Thr Phe Thr Ser Asp Leu Ser Lys Gln Leu Glu Glu Glu Ala Val Arg Leu Phe Ile Glu Phe Leu Lys Asn Gly Gly Pro Ser-NH₂ [SEQ. ID. NO. 177]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3477.9.

### EXAMPLE 173

### Preparation of Peptide Having SEQ ID NO. 178

His Gly Glu Ala Thr Phe Thr Ser Asp Leu Ser Lys Gln Met Glu Glu Glu Ala Val Arg Leu Phe Ile Glu Trp Leu Lys Asn Gly Gly Pro-NH₂ [SEQ. ID. NO. 178]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3519.9.

### EXAMPLE 174

### Preparation of Peptide Having SEQ ID NO. 179

His Gly Glu Gly Thr Phe Thr Ser Ala Leu Ser Lys Gln Leu Glu Glu Glu Ala Val Arg Leu Phe Ile Glu Phe Leu Lys Asn Gly Gly-NH₂ [SEQ. ID. NO. 179]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3307.7.

### EXAMPLE 175

### Preparation of Peptide Having SEQ ID NO. 180

Ala Gly Glu Gly Thr Phe Thr Ser Asp Leu Ser Lys Gln Leu Glu Glu Glu Ala Val Arg Leu Phe Ile Glu Phe Leu Lys Asn Gly-NH₂ [SEQ. ID. NO. 180]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3186.5.

### EXAMPLE 176

### Preparation of Peptide Having SEQ ID NO. 181

His Gly Ala Gly Thr Phe Thr Ser Asp Leu Ser Lys Gln Met Glu Glu Glu Ala Val Arg Leu Phe Ile Glu Trp Leu Lys Asn Gly Gly tPro Ser Ser Gly Ala tPro tPro tPro-NH₂ [SEQ. ID. NO. 181]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Double couplings are required at residues 37,36 and 31. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4121.1.

### EXAMPLE 177

### Preparation of Peptide Having SEQ ID NO. 182

His Gly Glu Ala Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala tPro tPro tPro-NH₂ [SEQ. ID. NO. 182].

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Double couplings are required at residues 37, 36 and 31. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4173.2.

### EXAMPLE 178

### Preparation of Peptide Having SEQ ID NO. 183

His Gly Glu Gly Thr Phe Thr Ser Ala Leu Ser Lys Gln Met Glu Glu Glu Ala Val Arg Leu Phe Ile Glu Trp Leu Lys Asn Gly Gly NMeala Ser Ser Gly Ala NMeala NMeala-NH₂ [SEQ. ID. NO. 183]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Double couplings are required at residues 36 and 31. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3796.1.

### EXAMPLE 179

### Preparation of Peptide Having SEQ ID NO. 184

Ala Gly Glu Gly Thr Phe Thr Ser Asp Leu Ser Lys Gln Met Glu Glu Glu Ala Val Arg Leu Phe Ile Glu Trp Leu Lys Asn Gly Gly hPro Ser Ser Gly Ala hPro-NH₂ [SEQ. ID. NO. 184]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. A double coupling is required at residue 31. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electro spray Mass Spectrometry(M): calculated 3871.1.

### EXAMPLE 180

### Preparation of Peptide Having SEQ ID NO. 185

His Gly Ala Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala-NH₂ [SEQ. ID. NO. 185]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3750.2.

### EXAMPLE 181

### Preparation of Peptide Having SEQ ID NO. 186

His Gly Asp Ala Thr Phe Thr Ser Asp Leu Ser Lys Gln Met Glu Glu Glu Ala Val Arg Leu Phe Ile Glu Trp Leu Lys Asn Gly Gly-NH₂ [SEQ. ID. NO. 186]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 3408.8.

### EXAMPLE 182

### Preparation of Peptide Having SEQ ID NO. 187

Ala Gly Glu Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala Pro Pro Pro Ser-NH₂ [SEQ. ID. NO. 187]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4120.6.

### EXAMPLE 183

### Preparation of Peptide Having SEQ ID NO. 188

Ala Gly Ala Gly Thr Phe Thr Ser Asp Leu Ser Lys Gln Leu Glu Glu Glu Ala Val Arg Leu Phe Ile Glu Phe Leu Lys Asn Gly Gly Pro Ser Ser Gly Ala Pro Pro Pro Ser-NH₂ [SEQ. ID. NO. 188]

The above-identified amidated peptide is assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.), cleaved from the resin, deprotected and purified in a similar way to Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry (M): calculated 4005.5.

### EXAMPLE 184

### Preparation of C-terminal carboxylic Acid Peptides Corresponding to the Above C-terminal Amide Sequences for Peptides Having SEQ ID NOS. 100-166, 172-177, 179-180 and 185-188.

C-terminal carboxylic acid peptides corresponding to amidated having SEQ ID NOS. 100-166, 172-177, 179-180 and 185-188 are assembled on the so called Wang resin (p-alkoxybenzylalacohol resin (Bachem, 0.54 mmole/g)) using Fmoc-protected amino acids (Applied Biosystems, Inc.), cleaved from the resin, deprotected and purified in a similar way to that described in Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry provides an experimentally determined (M).

### EXAMPLE 185

### Preparation of C-terminal Carboxylic Acid Peptides Corresponding to the Above C-terminal Amide Sequences for Peptides Having SEQ ID NOS. 167-171, 178 and 181-184.

C-terminal carboxylic acid peptides corresponding to amidated SEQ ID NOS. 167-171, 178 and 181-184 are assembled on the 2-chlorotritylchloride resin (200-400 mesh), 2% DVB (Novabiochem, 0.4-1.0 mmole/g)) using Fmoc-protected amino acids (Applied Biosystems, Inc.), cleaved from the resin, deprotected and purified in a similar way to that described in Example 95. Used in analysis are Solvent A (0.1% TFA in water) and Solvent B (0.1% TFA in ACN). Analytical RP-HPLC (gradient 30% to 60% Solvent B in Solvent A over 30 minutes) of the lyophilized peptide is then carried out to determine the retention time of the product peptide. Electrospray Mass Spectrometry provides an experimentally determined (M).

### EXAMPLE 186

### Evaluation of Ability to Lower Triglycerides in Humans

The safety, tolerability, and efficacy of synthetic exendin-4 was evaluated in 24 patients with type 2 diabetes previously treated by diet, oral hypoglycemic agents (OHA), or insulin in a single blind, placebo controlled, two period crossover study. The study compared the effects of multiple doses of synthetic exendin-4 and placebo given twice daily (before breakfast and dinner) for five days. Following screening, subjects were randomly assigned to receive synthetic exendin-4 or placebo for five days. After a two to three day washout period subjects crossed over and received the alternate therapy for the next five days.

Fourteen days prior to randomization, OHA therapy was stopped and subjects using insulin were stabilized on a single hs NPH injection for use during the study. Each patient was randomized to receive subcutaneous injections (BID) of placebo or 0.1 μg/kg synthetic exendin-4 for five days. Following a 2-3 day washout, subjects were randomly crossed over to the other treatment. Plasma glucose, glucagon, and serum triglyceride concentrations were assessed fasting and in response to a 7 Kcal/kg Sustacal® meal administered at the time of the AM synthetic exendin-4/placebo injection on days 1 and 5. Gastric emptying was evaluated by inclusion of 20 mg/kg liquid acetaminophen (ACET) with the Sustacal® meal and measuring serum ACET concentrations. Reported adverse events, EKG, physical exam, and safety lab monitoring revealed no safety issues. Nausea, vomiting, and hypoglycemia were the most frequent adverse events, however all were reported as mild in intensity.

Importantly, postprandial circulating triglycerides, plasma glucose, and glucagon were significantly reduced following synthetic exendin-4 compared to placebo on both days 1 and 5.

On day 5, the 5 hour time-weighted mean±SE change in plasma glucose from baseline was −7.7±5.1 mg/dL for AC2993 compared to 67.2±7.9 mg/dL for placebo (P<0.0001).

The 3 hour postprandial plasma glucagon area under the curve (AUC) was reduced by 23% compared to placebo (P=0.0123) and peak postprandial triglyceride concentrations were reduced 24% compared to placebo (P=0.0001).

The 5-hr mean total ACET was reduced by 57% compared to PBO, indicating a slowing of gastric emptying. In summary, subcutaneous injection of 0.1μg/kg synthetic exendin-4 in patients with type 2 diabetes identified no safety issues, reduced circulating postprandial triglyceride, plasma glucose, and glucagon concentrations, and slowed gastric emptying.

Various modifications of the invention in addition to those shown and described herein will become apparent to those skilled in the art from the foregoing description and fall within the scope of the following claims.

[image]

We claim:

1. A method for moduating triglyceride levels in a subject comprising administering to said subject a therapeutically effective amount of an exendin or an exendin agonist.

2. The method according to claim 1 wherein said exendin or exendin agonist is administered continuously.

3. The method according to claim 1 wherein said administration is by injection.

4. The method according to claim 3 wherein the injection is a subcutaneous injection.

5. The method according to claim 1 wherein about 1 μg-30μg to about 1 mg of the exendin or exendin agonist is administered per day.

6. The method according to claim 1 wherein about 1 μg-30 μg to about 500 μg of the exendin or exendin agonist is administered per day.

7. The method according to claim 1 wherein about 1 μg-30 μg to about 100 μg of the exendin or exendin agonist is administered per day.

8. The method according to claim 1, wherein about 3 μg to about 50 μg of the exendin or exendin agonist is administered per day.

9. The method of claim 1 wherein said subject is human.

10. A method for modulating plasma triglyceride concentrations in a subject with heart disease comprising administering to said subject a therapeutically effective amount of an exendin or an exendin agonist.

11. The method according to any of claims 1-10 wherein said exendin is exendin-3.

12. The method according to any of claims 1-10 wherein said exendin is exendin-4.

13. The method according to any of claims 1-10 wherein said exendin agonist is selected from the group consisting of exendin-4 acid, exendin-4 (1-30), exendin-4 (1-30) amide, exendin-4 (1-28) amide, ¹⁴Leu,²⁵Phe exendin-4, and ¹⁴Leu,²⁵Phe exendin-4 (1-28) amide.

14. The method according to any of claims 1-10 wherein said exendin agonist is an exendin analog or derivative.

15. The method according to any of claims 1-10, further comprising administering a therapeutically effective amount of a statin.

16. The method according to any of claims 1-10 wherein said exendin agonist is an exendin agonist according to Formula I.

17. The method according to any of claims 1-10 wherein said exendin agonist is an exendin agonist according to Formula II.

18. The method according to any of claims 1-10 wherein said exendin agonist is an exendin agonist according to Formula III.

19. A method for treating dyslipidemia in a subject comprising administering to said subject a therapeutically effective amount of an exendin or an exendin agonist.

20. The method according to claim 19 wherein said exendin agonist is an exendin analog or derivative.

21. The method according to claim 19 wherein said exendin agonist is an exendin agonist according to Formmula I.

22. The method according to claim 19 wherein said exendin agonist is an exendin agonist according to Formula II.

23. The method according to claim 19 wherein said exendin agonist is an exendin agonist according to Formula III.

24. A method for modulating postprandial triglyceride levels in a subject comprising administering to said subject a therapeutically effective amount of an exendin or an exendin agonist.

25. The method according to claim 24 wherein said exendin or exendin agonist is administered continuously.

26. The method according to claim 24 wherein said administration is by injection.

27. The method according to claim 26 wherein the injection is a subcutaneous injection.

28. The method according to claim 24 wherein about 1 μg-30μg to about 1 mg of the exendin or exendin agonist is administered per day.

29. The method according to claim 24 wherein about 1 μg-30 μg to about 500 μg of the exendin or exendin agonist is administered per day.

30. The method according to claim 24 wherein about 1 μg-30 μg to about 100 μg of the exendin or exendin agonist is administered per day.

31. The method according to claim 24, wherein about 3 μg to about 50 μg of the exendin or exendin agonist is administered per day.

32. The method of claim 24 wherein said subject is human.

33. The method according to any of claims 24-32 wherein said exendin is exendin-3.

34. The method according to any of claims 24-32 wherein said exendin is exendin-4.

35. The method according to any of claims 24-32 wherein said exendin agonist is selected from the group consisting of exendin-4 acid, exendin-4 (1-30), exendin-4 (1-30) amide, exendin-4 (1-28) amide, ¹⁴Leu,²⁵Phe exendin-4, and ⁴Leu,²⁵Phe exendin-4 (1-28)amide.

36. The method according to any of claims 24-32 wherein said exendin agonist is an exendin analog or derivative.

37. The method according to any of claims 24-32, further comprising administering a therapeutically effective amount of a statin.

38. The method according to any of claims 24-32 wherein said exendin agonist is an exendin agonist according to Formula I.

39. The method according to any of claims 24-32 wherein said exendin agonist is an exendin agonist according to Formula II.

40. The method according to any of claims 24-32 wherein said exendin agonist is an exendin agonist according to Formula III.


SEQUENCE LISTING (188 sequences)

SEQ ID NO: 1 - protein, 39 aa, Heloderma horridum
  Exendin-3
HSDGTFTSDLSKQMEEEAVRLFIEWLKNGGPSSGAPPPS

SEQ ID NO: 2 - protein, 39 aa, Heloderma suspectum
  Exendin-4
HGEGTFTSDLSKQMEEEAVRLFIEWLKNGGPSSGAPPPS

SEQ ID NO: 3 - protein, 39 aa, Artificial Sequence
  Description of Artificial Sequence Exendin Agonist
XXXGTXXXXXSKQXEEEAVRLXXXXLKNGGXSSGAXXXX

SEQ ID NO: 4 - protein, 38 aa, Artificial Sequence
  Description of Artificial Sequence Exendin Agonist
XXXGXXXXXXXXXXXXXAXXXXXXXXXXGGXSSGAXXX

SEQ ID NO: 5 - protein, 39 aa, Artificial Sequence
  Description of Artificial Sequence Exendin Agonist
XXXXXXXXXXXXXXXXXAXXXXXXXXXXGGXSSGAXXXS

SEQ ID NO: 6 - protein, 30 aa, Artificial Sequence
  Description of Artificial Sequence Exendin Agonist
HGEGTFTSDLSKQMEEEAVRLFIEWLKNGG

SEQ ID NO: 7 - protein, 30 aa, Artificial Sequence; identical to SEQ ID NO: 6
  Description of Artificial Sequence Exendin Agonist

SEQ ID NO: 8 - protein, 28 aa, Artificial Sequence
  Description of Artificial Sequence Exendin Agonist
HGEGTFTSDLSKQLEEEAVRLAIEFLKN

SEQ ID NO: 9 - protein, 39 aa, Artificial Sequence
  Description of Artificial Sequence Exendin Agonist
HGEGTFTSDLSKQLEEEAVRLFIEFLKNGGPSSGAPPPS

SEQ ID NO: 10 - protein, 39 aa, Artificial Sequence
  Description of Artificial Sequence Exendin Agonist
HGEGTFTSDLSKQLEEEAVRLFIEWLKNGGPSSGAPPPS

SEQ ID NO: 11 - protein, 38 aa, Artificial Sequence
  Description of Artificial Sequence Exendin Agonist
HGEGTFTSDLSKQMEEEAVRFIEFLKNGGPSSGAPPPS

SEQ ID NO: 12 - protein, 39 aa, Artificial Sequence
  Description of Artificial Sequence Exendin Agonist
YGEGTFTSDLSKQMEEEAVRLFIEWLKNGGPSSGAPPPS

SEQ ID NO: 13 - protein, 39 aa, Artificial Sequence
  Description of Artificial Sequence Exendin Agonist
HGEGTFTSDLSKQMEEEAVRLFIEWLKNGGPSSGAPPPY

SEQ ID NO: 14 - protein, 39 aa, Artificial Sequence
  Description of Artificial Sequence Exendin Agonist
HGDGTFTSDLSKQMEEEAVRLFIEWLKNGGPSSGAPPPS

SEQ ID NO: 15 - protein, 42 aa, Artificial Sequence
  Description of Artificial Sequence Exendin Agonist
HGEGTXTSDLSDLSKQMEEEAVRLFIEWLKNGGPSSGAPPPS

SEQ ID NO: 16 - protein, 39 aa, Artificial Sequence
  Description of Artificial Sequence Exendin Agonist
HGEGTFSSDLSKQMEEEAVRLFIEWLKNGGPSSGAPPPS

SEQ ID NO: 17 - protein, 39 aa, Artificial Sequence
  Description of Artificial Sequence Exendin Agonist
HGEGTFSTDLSKQMEEEAVRLFIEWLKNGGPSSGAPPPS

SEQ ID NO: 18 - protein, 39 aa, Artificial Sequence
  Description of Artificial Sequence Exendin Agonist
HGEGTFTTDLSKQMEEEAVRLFIEWLKNGGPSSGAPPPS

SEQ ID NO: 19 - protein, 39 aa, Artificial Sequence
  Description of Artificial Sequence Exendin Agonist
HGEGTFTSELSKQMEEEAVRLFIEWLKNGGPSSGAPPPS

SEQ ID NO: 20 - protein, 39 aa, Artificial Sequence
  Description of Artificial Sequence Exendin Agonist
HGEGTFTSDXSKQMEEEAVRLFIEWLKNGGPSSGAPPPS

SEQ ID NO: 21 - protein, 39 aa, Artificial Sequence
  Description of Artificial Sequence Exendin Agonist
HGEGTFTSDXSKQLEEEAVRLFIEFLKNGGPSSGAPPPS

SEQ ID NO: 22 - protein, 39 aa, Artificial Sequence
  Description of Artificial Sequence Exendin Agonist
HGEGTFTSDLSKQXEEEAVRLFIEWLKNGGPSSGAPPPS

SEQ ID NO: 23 - protein, 39 aa, Artificial Sequence
  Description of Artificial Sequence Exendin Agonist
HGEGTFTSDLSKQXEEEAVRLFIEFLKNGGPSSGAPPPS

SEQ ID NO: 24 - protein, 39 aa, Artificial Sequence
  Description of Artificial Sequence Exendin Agonist
HGEGTFTSDLSKQMEEEAVRLXIEWLKNGGPSSGAPPPS

SEQ ID NO: 25 - protein, 39 aa, Artificial Sequence
  Description of Artificial Sequence Exendin Agonist
HGEGTFTSDLSKQMEEEAVRLFVEWLKNGGPSSGAPPPS

SEQ ID NO: 26 - protein, 39 aa, Artificial Sequence
  Description of Artificial Sequence Exendin Agonist
HGEGTFTSDLSKQLEEEAVRLFVEFLKNGGPSSGAPPPS

SEQ ID NO: 27 - protein, 39 aa, Artificial Sequence
  Description of Artificial Sequence Exendin Agonist
HGEGTFTSDLSKQMEEEAVRLFXEWLKNGGPSSGAPPPS

SEQ ID NO: 28 - protein, 39 aa, Artificial Sequence
  Description of Artificial Sequence Exendin Agonist
HGEGTFTSDLSKQLEEEAVRLFXEFLKNGGPSSGAPPPS

SEQ ID NO: 29 - protein, 39 aa, Artificial Sequence
  Description of Artificial Sequence Exendin Agonist
HGEGTFTSDLSKQMEEEAVRLFIDWLKNGGPSSGAPPPS

SEQ ID NO: 30 - protein, 39 aa, Artificial Sequence
  Description of Artificial Sequence Exendin Agonist
HGEGTFTSDLSKQMEEEAVRLFIEFLKNGGPSSGAPPPS

SEQ ID NO: 31 - protein, 39 aa, Artificial Sequence
  Description of Artificial Sequence Exendin Agonist
HGEGTFTSDLSKQMEEEAVRLFIEWLKNGGXSSGAXXXS

SEQ ID NO: 32 - protein, 39 aa, Artificial Sequence
  Description of Artificial Sequence Exendin Agonist
HGEGTFTSDLSKQMEEEAVRLFIEWLKNGGPSSGAXXXS

SEQ ID NO: 33 - protein, 39 aa, Artificial Sequence; identical to SEQ ID NO: 31
  Description of Artificial Sequence Exendin Agonist

SEQ ID NO: 34 - protein, 39 aa, Artificial Sequence; identical to SEQ ID NO: 32
  Description of Artificial Sequence Exendin Agonist

SEQ ID NO: 35 - protein, 39 aa, Artificial Sequence
  Description of Artificial Sequence Exendin Agonist
HGEGTFTSDLSKQLEEEAVRLFIEFLKNGGXSSGAXXXS

SEQ ID NO: 36 - protein, 39 aa, Artificial Sequence; identical to SEQ ID NO: 35
  Description of Artificial Sequence Exendin Agonist

SEQ ID NO: 37 - protein, 39 aa, Artificial Sequence; identical to SEQ ID NO: 31
  Description of Artificial Sequence Exendin Agonist

SEQ ID NO: 38 - protein, 39 aa, Artificial Sequence; identical to SEQ ID NO: 32
  Description of Artificial Sequence Exendin Agonist

SEQ ID NO: 39 - protein, 39 aa, Artificial Sequence; identical to SEQ ID NO: 35
  Description of Artificial Sequence Exendin Agonist

SEQ ID NO: 40 - protein, 28 aa, Artificial Sequence
  Description of Artificial Sequence Exendin Agonist
HGEGTFTSDLSKQMEEEAVRLFIEWLKN

SEQ ID NO: 41 - protein, 28 aa, Artificial Sequence
  Description of Artificial Sequence Exendin Agonist
HGEGTFTSDLSKQLEEEAVRLFIEFLKN

SEQ ID NO: 42 - protein, 28 aa, Artificial Sequence
  Description of Artificial Sequence Exendin Agonist
HAEGTFTSDLSKQLEEEAVRLFIEFLKN

SEQ ID NO: 43 - protein, 28 aa, Artificial Sequence
  Description of Artificial Sequence Exendin Agonist
HGEGAFTSDLSKQLEEEAVRLFIEFLKN

SEQ ID NO: 44 - protein, 28 aa, Artificial Sequence
  Description of Artificial Sequence Exendin Agonist
HGEGTATSDLSKQLEEEAVRLFIEFLKN

SEQ ID NO: 45 - protein, 28 aa, Artificial Sequence
  Description of Artificial Sequence Exendin Agonist
HGEGTFTADLSKQLEEEAVRLFIEFLKN

SEQ ID NO: 46 - protein, 28 aa, Artificial Sequence
  Description of Artificial Sequence Exendin Agonist
HGEGTFTSDASKQLEEEAVRLFIEFLKN

SEQ ID NO: 47 - protein, 28 aa, Artificial Sequence
  Description of Artificial Sequence Exendin Agonist
HGEGTFTSDLAKQLEEEAVRLFIEFLKN

SEQ ID NO: 48 - protein, 28 aa, Artificial Sequence
  Description of Artificial Sequence Exendin Agonist
HGEGTFTSDLSAQLEEEAVRLFIEFLKN

SEQ ID NO: 49 - protein, 28 aa, Artificial Sequence
  Description of Artificial Sequence Exendin Agonist
HGEGTFTSDLSKALEEEAVRLFIEFLKN

SEQ ID NO: 50 - protein, 28 aa, Artificial Sequence
  Description of Artificial Sequence Exendin Agonist
HGEGTFTSDLSKQAEEEAVRLFIEFLKN

SEQ ID NO: 51 - protein, 28 aa, Artificial Sequence
  Description of Artificial Sequence Exendin Agonist
HGEGTFTSDLSKQLAEEAVRLFIEFLKN

SEQ ID NO: 52 - protein, 28 aa, Artificial Sequence
  Description of Artificial Sequence Exendin Agonist
HGEGTFTSDLSKQLEAEAVRLFIEFLKN

SEQ ID NO: 53 - protein, 28 aa, Artificial Sequence
  Description of Artificial Sequence Exendin Agonist
HGEGTFTSDLSKQLEEAAVRLFIEFLKN

SEQ ID NO: 54 - protein, 28 aa, Artificial Sequence
  Description of Artificial Sequence Exendin Agonist
HGEGTFTSDLSKQLEEEAARLFIEFLKN

SEQ ID NO: 55 - protein, 28 aa, Artificial Sequence
  Description of Artificial Sequence Exendin Agonist
HGEGTFTSDLSKQLEEEAVALFIEFLKN

SEQ ID NO: 56 - protein, 28 aa, Artificial Sequence
  Description of Artificial Sequence Exendin Agonist
HGEGTFTSDLSKQLEEEAVRAFIEFLKN

SEQ ID NO: 57 - protein, 28 aa, Artificial Sequence
  Description of Artificial Sequence Exendin Agonist
HGEGTFTSDLSKQLEEEAVRLFIAFLKN

SEQ ID NO: 58 - protein, 28 aa, Artificial Sequence
  Description of Artificial Sequence Exendin Agonist
HGEGTFTSDLSKQLEEEAVRLFIEALKN

SEQ ID NO: 59 - protein, 28 aa, Artificial Sequence
  Description of Artificial Sequence Exendin Agonist
HGEGTFTSDLSKQLEEEAVRLFIEFAKN

SEQ ID NO: 60 - protein, 28 aa, Artificial Sequence
  Description of Artificial Sequence Exendin Agonist
HGEGTFTSDLSKQLEEEAVRLFIEFLAN

SEQ ID NO: 61 - protein, 28 aa, Artificial Sequence
  Description of Artificial Sequence Exendin Agonist
HGEGTFTSDLSKQLEEEAVRLFIEFLKA

SEQ ID NO: 62 - protein, 38 aa, Artificial Sequence
  Description of Artificial Sequence Exendin Agonist
HGEGTFTSDLSKQMEEEAVRLFIEWLKNGGPSSGAPPP

SEQ ID NO: 63 - protein, 38 aa, Artificial Sequence
  Description of Artificial Sequence Exendin Agonist
HGEGTFTSDLSKQLEEEAVRLFIEFLKNGGPSSGAPPP

SEQ ID NO: 64 - protein, 37 aa, Artificial Sequence
  Description of Artificial Sequence Exendin Agonist
HGEGTFTSDLSKQMEEEAVRLFIEWLKNGGPSSGAPP

SEQ ID NO: 65 - protein, 37 aa, Artificial Sequence
  Description of Artificial Sequence Exendin Agonist
HGEGTFTSDLSKQLEEEAVRLFIEFLKNGGPSSGAPP

SEQ ID NO: 66 - protein, 36 aa, Artificial Sequence
  Description of Artificial Sequence Exendin Agonist
HGEGTFTSDLSKQMEEEAVRLFIEWLKNGGPSSGAP

SEQ ID NO: 67 - protein, 36 aa, Artificial Sequence
  Description of Artificial Sequence Exendin Agonist
HGEGTFTSDLSKQLEEEAVRLFIEFLKNGGPSSGAP

SEQ ID NO: 68 - protein, 35 aa, Artificial Sequence
  Description of Artificial Sequence Exendin Agonist
HGEGTFTSDLSKQMEEEAVRLFIEWLKNGGPSSGA

SEQ ID NO: 69 - protein, 35 aa, Artificial Sequence
  Description of Artificial Sequence Exendin Agonist
HGEGTFTSDLSKQLEEEAVRLFIEFLKNGGPSSGA

SEQ ID NO: 70 - protein, 34 aa, Artificial Sequence
  Description of Artificial Sequence Exendin Agonist
HGEGTFTSDLSKQMEEEAVRLFIEWLKNGGPSSG

SEQ ID NO: 71 - protein, 34 aa, Artificial Sequence
  Description of Artificial Sequence Exendin Agonist
HGEGTFTSDLSKQLEEEAVRLFIEFLKNGGPSSG

SEQ ID NO: 72 - protein, 33 aa, Artificial Sequence
  Description of Artificial Sequence Exendin Agonist
HGEGTFTSDLSKQMEEEAVRLFIEWLKNGGPSS

SEQ ID NO: 73 - protein, 33 aa, Artificial Sequence
  Description of Artificial Sequence Exendin Agonist
HGEGTFTSDLSKQLEEEAVRLFIEFLKNGGPSS

SEQ ID NO: 74 - protein, 32 aa, Artificial Sequence
  Description of Artificial Sequence Exendin Agonist
HGEGTFTSDLSKQMEEEAVRLFIEWLKNGGPS

SEQ ID NO: 75 - protein, 32 aa, Artificial Sequence
  Description of Artificial Sequence Exendin Agonist
HGEGTFTSDLSKQLEEEAVRLFIEFLKNGGPS

SEQ ID NO: 76 - protein, 31 aa, Artificial Sequence
  Description of Artificial Sequence Exendin Agonist
HGEGTFTSDLSKQMEEEAVRLFIEWLKNGGP

SEQ ID NO: 77 - protein, 31 aa, Artificial Sequence
  Description of Artificial Sequence Exendin Agonist
HGEGTFTSDLSKQLEEEAVRLFIEFLKNGGP

SEQ ID NO: 78 - protein, 30 aa, Artificial Sequence
  Description of Artificial Sequence Exendin Agonist
HGEGTFTSDLSKQLEEEAVRLFIEFLKNGG

SEQ ID NO: 79 - protein, 29 aa, Artificial Sequence
  Description of Artificial Sequence Exendin Agonist
HGEGTFTSDLSKQMEEEAVRLFIEWLKNG

SEQ ID NO: 80 - protein, 29 aa, Artificial Sequence
  Description of Artificial Sequence Exendin Agonist
HGEGTFTSDLSKQLEEEAVRLFIEFLKNG

SEQ ID NO: 81 - protein, 38 aa, Artificial Sequence
  Description of Artificial Sequence Exendin Agonist
HGEGTFTSDLSKQMEEEAVRLFIEWLKNGGXSSGAXXX

SEQ ID NO: 82 - protein, 38 aa, Artificial Sequence
  Description of Artificial Sequence Exendin Agonist
HGEGTFTSDLSKQMEEEAVRLFIEWLKNGGPSSGAXXX

SEQ ID NO: 83 - protein, 37 aa, Artificial Sequence
  Description of Artificial Sequence Exendin Agonist
HGEGTFTSDLSKQMEEEAVRLFIEWLKNGGXSSGAPP

SEQ ID NO: 84 - protein, 37 aa, Artificial Sequence
  Description of Artificial Sequence Exendin Agonist
HGEGTFTSDLSKQMEEEAVRLFIEWLKNGGXSSGAXX

SEQ ID NO: 85 - protein, 37 aa, Artificial Sequence; identical to SEQ ID NO: 84
  Description of Artificial Sequence Exendin Agonist

SEQ ID NO: 86 - protein, 36 aa, Artificial Sequence
  Description of Artificial Sequence Exendin Agonist
HGEGTFTSDLSKQMEEEAVRLFIEWLKNGGXSSGAX

SEQ ID NO: 87 - protein, 35 aa, Artificial Sequence
  Description of Artificial Sequence Exendin Agonist
RGEGTFTSDLSKQMEEEAVRLFIEWLKNGGPSSGA

SEQ ID NO: 88 - protein, 30 aa, Artificial Sequence
  Description of Artificial Sequence Exendin Agonist
HGDGTFTSDLSKQMEEEAVRLFIEWLKNGG

SEQ ID NO: 89 - protein, 28 aa, Artificial Sequence
  Description of Artificial Sequence Exendin Agonist
HGEGTXTSDLSKQLEEEAVRLFIEFLKN

SEQ ID NO: 90 - protein, 28 aa, Artificial Sequence
  Description of Artificial Sequence Exendin Agonist
HGEGTFSSDLSKQMEEEAVRLFIEWLKN

SEQ ID NO: 91 - protein, 28 aa, Artificial Sequence
  Description of Artificial Sequence Exendin Agonist
HGEGTFSTDLSKQMEEEAVRLFIEWLKN

SEQ ID NO: 92 - protein, 28 aa, Artificial Sequence
  Description of Artificial Sequence Exendin Agonist
HGEGTFTSELSKQMAEEAVRLFIEWLKN

SEQ ID NO: 93 - protein, 28 aa, Artificial Sequence
  Description of Artificial Sequence Exendin Agonist
HGEGTFTSDXSKQLEEEAVRLFIEFLKN

SEQ ID NO: 94 - protein, 28 aa, Artificial Sequence
  Description of Artificial Sequence Exendin Agonist
HGEGTFTSDLSKQLEEEAVRLXIEFLKN

SEQ ID NO: 95 - protein, 28 aa, Artificial Sequence
  Description of Artificial Sequence Exendin Agonist
HGEGTFTSDLSKQMEEEAVRLFXEWLKN

SEQ ID NO: 96 - protein, 28 aa, Artificial Sequence
  Description of Artificial Sequence Exendin Agonist
HGEGTFTSDLSKQLEEEAVRLFIDFLKN

SEQ ID NO: 97 - protein, 33 aa, Artificial Sequence
  Description of Artificial Sequence Exendin Agonist
HGEGTFTSDASKQLEEEAVRLFIEFLKNGGPSS

SEQ ID NO: 98 - protein, 29 aa, Artificial Sequence
  Description of Artificial Sequence Exendin Agonist
HGEGTFTSDASKQMEEEAVRLFIEWLKNG

SEQ ID NO: 99 - protein, 37 aa, Artificial Sequence
  Description of Artificial Sequence Exendin Agonist
HGEGTFTSDASKQMEEEAVRLFIEWLKNGGXSSGAXX

SEQ ID NO: 100 - protein, 28 aa, Artificial Sequence
  Description of Artificial Sequence Exendin Agonist
AGEGTFTSDLSKQLEEEAVRLFIEFLKN

SEQ ID NO: 101 - protein, 28 aa, Artificial Sequence
  Description of Artificial Sequence Exendin Agonist
HGAGTFTSDLSKQLEEEAVRLFIEFLKN

SEQ ID NO: 102 - protein, 28 aa, Artificial Sequence
  Description of Artificial Sequence Exendin Agonist
HGEATFTSDLSKQLEEEAVRLFIEFLKN

SEQ ID NO: 103 - protein, 28 aa, Artificial Sequence
  Description of Artificial Sequence Exendin Agonist
HGEGTFTSALSKQLEEEAVRLFIEFLKN

SEQ ID NO: 104 - protein, 28 aa, Artificial Sequence
  Description of Artificial Sequence Exendin Agonist
AGEGTFTSDLSKQMEEEAVRLFIEWLKN

SEQ ID NO: 105 - protein, 28 aa, Artificial Sequence
  Description of Artificial Sequence Exendin Agonist
HGAGTFTSDLSKQMEEEAVRLFIEWLKN

SEQ ID NO: 106 - protein, 28 aa, Artificial Sequence
  Description of Artificial Sequence Exendin Agonist
HGEATFTSDLSKQMEEEAVRLFIEWLKN

SEQ ID NO: 107 - protein, 28 aa, Artificial Sequence
  Description of Artificial Sequence Exendin Agonist
HGEGTFTSALSKQMEEEAVRLFIEWLKN

SEQ ID NO: 108 - protein, 28 aa, Artificial Sequence
  Description of Artificial Sequence Exendin Agonist
HGEGTFTSDASKQMEEEAVRLFIEWLKN

SEQ ID NO: 109 - protein, 28 aa, Artificial Sequence
  Description of Artificial Sequence Exendin Agonist
AAEGTFTSDLSKQMEEEAVRLFIEWLKN

SEQ ID NO: 110 - protein, 28 aa, Artificial Sequence
  Description of Artificial Sequence Exendin Agonist
AAEGTFTSDLSKQLEEEAVRLFIEFLKN

SEQ ID NO: 111 - protein, 28 aa, Artificial Sequence
  Description of Artificial Sequence Exendin Agonist
AGDGTFTSDLSKQMEEEAVRLFIEWLKN

SEQ ID NO: 112 - protein, 28 aa, Artificial Sequence
  Description of Artificial Sequence Exendin Agonist
AGDGTFTSDLSKQLEEEAVRLFIEFLKN

SEQ ID NO: 113 - protein, 28 aa, Artificial Sequence
  Description of Artificial Sequence Exendin Agonist
AGDGAFTSDLSKQMEEEAVRLFIEWLKN

SEQ ID NO: 114 - protein, 28 aa, Artificial Sequence
  Description of Artificial Sequence Exendin Agonist
AGDGAFTSDLSKQLEEEAVRLFIEFLKN

SEQ ID NO: 115 - protein, 28 aa, Artificial Sequence
  Description of Artificial Sequence Exendin Agonist
AGDGTXTSDLSKQMEEEAVRLFIEWLKN

SEQ ID NO: 116 - protein, 28 aa, Artificial Sequence
  Description of Artificial Sequence Exendin Agonist
AGDGTXTSDLSKQLEEEAVRLFIEFLKN

SEQ ID NO: 117 - protein, 28 aa, Artificial Sequence
  Description of Artificial Sequence Exendin Agonist
AGDGTFSSDLSKQMEEEAVRLFIEWLKN

SEQ ID NO: 118 - protein, 28 aa, Artificial Sequence
  Description of Artificial Sequence Exendin Agonist
AGDGTFSSDLSKQLEEEAVRLFIEFLKN

SEQ ID NO: 119 - protein, 28 aa, Artificial Sequence
  Description of Artificial Sequence Exendin Agonist
AGDGTFTADLSKQMEEEAVRLFIEWLKN

SEQ ID NO: 120 - protein, 28 aa, Artificial Sequence
  Description of Artificial Sequence Exendin Agonist
AGDGTFTADLSKQLEEEAVRLFIEFLKN

SEQ ID NO: 121 - protein, 28 aa, Artificial Sequence
  Description of Artificial Sequence Exendin Agonist
AGDGTFTSALSKQMEEEAVRLFIEWLKN

SEQ ID NO: 122 - protein, 28 aa, Artificial Sequence
  Description of Artificial Sequence Exendin Agonist
AGDGTFTSALSKQLEEEAVRLFIEFLKN

SEQ ID NO: 123 - protein, 28 aa, Artificial Sequence
  Description of Artificial Sequence Exendin Agonist
AGDGTFTSELSKQMEEEAVRLFIEWLKN

SEQ ID NO: 124 - protein, 28 aa, Artificial Sequence
  Description of Artificial Sequence Exendin Agonist
AGDGTFTSELSKQLEEEAVRLFIEFLKN

SEQ ID NO: 125 - protein, 28 aa, Artificial Sequence
  Description of Artificial Sequence Exendin Agonist
AGDGTFTSDASKQMEEEAVRLFIEWLKN

SEQ ID NO: 126 - protein, 28 aa, Artificial Sequence
  Description of Artificial Sequence Exendin Agonist
AGDGTFTSDASKQLEEEAVRLFIEFLKN

SEQ ID NO: 127 - protein, 28 aa, Artificial Sequence
  Description of Artificial Sequence Exendin Agonist
AGDGTFTSDXSKQMEEEAVRLFIEWLKN

SEQ ID NO: 128 - protein, 28 aa, Artificial Sequence
  Description of Artificial Sequence Exendin Agonist
AGDGTFTSDXSKQLEEEAVRLFIEFLKN

SEQ ID NO: 129 - protein, 28 aa, Artificial Sequence
  Description of Artificial Sequence Exendin Agonist
AGDGTFTSDLAKQMEEEAVRLFIEWLKN

SEQ ID NO: 130 - protein, 28 aa, Artificial Sequence
  Description of Artificial Sequence Exendin Agonist
AGDGTFTSDLAKQLEEEAVRLFIEFLKN

SEQ ID NO: 131 - protein, 28 aa, Artificial Sequence
  Description of Artificial Sequence Exendin Agonist
AGDGTFTSDLSAQMEEEAVRLFIEWLKN

SEQ ID NO: 132 - protein, 28 aa, Artificial Sequence
  Description of Artificial Sequence Exendin Agonist
AGDGTFTSDLSAQLEEEAVRLFIEFLKN

SEQ ID NO: 133 - protein, 28 aa, Artificial Sequence
  Description of Artificial Sequence Exendin Agonist
AGDGTFTSDLSKAMEEEAVRLFIEWLKN

SEQ ID NO: 134 - protein, 28 aa, Artificial Sequence
  Description of Artificial Sequence Exendin Agonist
AGDGTFTSDLSKALEEEAVRLFIEFLKN

SEQ ID NO: 135 - protein, 28 aa, Artificial Sequence
  Description of Artificial Sequence Exendin Agonist
AGDGTFTSDLSKQAEEEAVRLFIEWLKN

SEQ ID NO: 136 - protein, 28 aa, Artificial Sequence
  Description of Artificial Sequence Exendin Agonist
AGDGTFTSDLSKQAEEEAVRLFIEFLKN

SEQ ID NO: 137 - protein, 28 aa, Artificial Sequence
  Description of Artificial Sequence Exendin Agonist
AGDGTFTSDLSKQXEEEAVRLFIEWLKN

SEQ ID NO: 138 - protein, 28 aa, Artificial Sequence
  Description of Artificial Sequence Exendin Agonist
AGDGTFTSDLSKQXEEEAVRLFIEFLKN

SEQ ID NO: 139 - protein, 28 aa, Artificial Sequence
  Description of Artificial Sequence Exendin Agonist
AGDGTFTSDLSKQMAEEAVRLFIEWLKN

SEQ ID NO: 140 - protein, 28 aa, Artificial Sequence
  Description of Artificial Sequence Exendin Agonist
AGDGTFTSDLSKQLAEEAVRLFIEFLKN

SEQ ID NO: 141 - protein, 28 aa, Artificial Sequence
  Description of Artificial Sequence Exendin Agonist
AGDGTFTSDLSKQMEAEAVRLFIEWLKN

SEQ ID NO: 142 - protein, 28 aa, Artificial Sequence
  Description of Artificial Sequence Exendin Agonist
AGDGTFTSDLSKQLEAEAVRLFIEFLKN

SEQ ID NO: 143 - protein, 28 aa, Artificial Sequence
  Description of Artificial Sequence Exendin Agonist
AGDGTFTSDLSKQMEEAAVRLFIEWLKN

SEQ ID NO: 144 - protein, 28 aa, Artificial Sequence
  Description of Artificial Sequence Exendin Agonist
AGDGTFTSDLSKQLEEAAVRLFIEFLKN

SEQ ID NO: 145 - protein, 28 aa, Artificial Sequence
  Description of Artificial Sequence Exendin Agonist
AGDGTFTSDLSKQMEEEAARLFIEWLKN

SEQ ID NO: 146 - protein, 28 aa, Artificial Sequence
  Description of Artificial Sequence Exendin Agonist
AGDGTFTSDLSKQLEEEAARLFIEFLKN

SEQ ID NO: 147 - protein, 28 aa, Artificial Sequence
  Description of Artificial Sequence Exendin Agonist
AGDGTFTSDLSKQMEEEAVALFIEWLKN

SEQ ID NO: 148 - protein, 28 aa, Artificial Sequence
  Description of Artificial Sequence Exendin Agonist
AGDGTFTSDLSKQLEEEAVALFIEFLKN

SEQ ID NO: 149 - protein, 28 aa, Artificial Sequence
  Description of Artificial Sequence Exendin Agonist
AGDGTFTSDLSKQMEEEAVRAFIEWLKN

SEQ ID NO: 150 - protein, 28 aa, Artificial Sequence
  Description of Artificial Sequence Exendin Agonist
AGDGTFTSDLSKQLEEEAVRAFIEFLKN

SEQ ID NO: 151 - protein, 28 aa, Artificial Sequence
  Description of Artificial Sequence Exendin Agonist
AGDGTFTSDLSKQMEEEAVRLXIEWLKN

SEQ ID NO: 152 - protein, 28 aa, Artificial Sequence
  Description of Artificial Sequence Exendin Agonist
AGDGTFTSDLSKQLEEEAVRLXIEFLKN

SEQ ID NO: 153 - protein, 28 aa, Artificial Sequence
  Description of Artificial Sequence Exendin Agonist
AGDGTFTSDLSKQMEEEAVRLFVEWLKN

SEQ ID NO: 154 - protein, 28 aa, Artificial Sequence
  Description of Artificial Sequence Exendin Agonist
AGDGTFTSDLSKQLEEEAVRLFVEFLKN

SEQ ID NO: 155 - protein, 28 aa, Artificial Sequence
  Description of Artificial Sequence Exendin Agonist
AGDGTFTSDLSKQMEEEAVRLFXEWLKN

SEQ ID NO: 156 - protein, 28 aa, Artificial Sequence
  Description of Artificial Sequence Exendin Agonist
AGDGTFTSDLSKQLEEEAVRLFXEFLKN

SEQ ID NO: 157 - protein, 28 aa, Artificial Sequence
  Description of Artificial Sequence Exendin Agonist
AGDGTFTSDLSKQMEEEAVRLFIDWLKN

SEQ ID NO: 158 - protein, 28 aa, Artificial Sequence
  Description of Artificial Sequence Exendin Agonist
AGDGTFTSDLSKQLEEEAVRLFIDFLKN

SEQ ID NO: 159 - protein, 28 aa, Artificial Sequence
  Description of Artificial Sequence Exendin Agonist
AGDGTFTSDLSKQMEEEAVRLFIEALKN

SEQ ID NO: 160 - protein, 28 aa, Artificial Sequence
  Description of Artificial Sequence Exendin Agonist
AGDGTFTSDLSKQLEEEAVRLFIEALKN

SEQ ID NO: 161 - protein, 28 aa, Artificial Sequence
  Description of Artificial Sequence Exendin Agonist
AGDGTFTSDLSKQMEEEAVRLFIEWAKN

SEQ ID NO: 162 - protein, 28 aa, Artificial Sequence
  Description of Artificial Sequence Exendin Agonist
AGDGTFTSDLSKQLEEEAVRLFIEFAKN

SEQ ID NO: 163 - protein, 28 aa, Artificial Sequence
  Description of Artificial Sequence Exendin Agonist
AGDGTFTSDLSKQMEEEAVRLFIEWLAN

SEQ ID NO: 164 - protein, 28 aa, Artificial Sequence
  Description of Artificial Sequence Exendin Agonist
AGDGTFTSDLSKQLEEEAVRLFIEFLAN

SEQ ID NO: 165 - protein, 28 aa, Artificial Sequence
  Description of Artificial Sequence Exendin Agonist
AGDGTFTSDLSKQMEEEAVRLFIEWLKA

SEQ ID NO: 166 - protein, 28 aa, Artificial Sequence
  Description of Artificial Sequence Exendin Agonist
AGDGTFTSDLSKQLEEEAVRLFIEFLKA

SEQ ID NO: 167 - protein, 38 aa, Artificial Sequence
  Description of Artificial Sequence Exendin Agonist
AGEGTFTSDLSKQMEEEAVRLFIEWLKNGGPSSGAPPP

SEQ ID NO: 168 - protein, 38 aa, Artificial Sequence
  Description of Artificial Sequence Exendin Agonist
HGAGTFTSDLSKQLEEEAVRLFIEFLKNGGPSSGAPPP

SEQ ID NO: 169 - protein, 37 aa, Artificial Sequence
  Description of Artificial Sequence Exendin Agonist
HGEATFTSDLSKQMEEEAVRLFIEWLKNGGPSSGAPP

SEQ ID NO: 170 - protein, 36 aa, Artificial Sequence
  Description of Artificial Sequence Exendin Agonist
HGEGTFTSALSKQMEEEAVRLFIEWLKNGGPSSGAP

SEQ ID NO: 171 - protein, 36 aa, Artificial Sequence
  Description of Artificial Sequence Exendin Agonist
AGEGTFTSDASKQLEEEAVRLFIEFLKNGGPSSGAP

SEQ ID NO: 172 - protein, 35 aa, Artificial Sequence
  Description of Artificial Sequence Exendin Agonist
AGEGTFTSDLSKQMEEEAVRLFIEWLKNGGPSSGA

SEQ ID NO: 173 - protein, 35 aa, Artificial Sequence
  Description of Artificial Sequence Exendin Agonist
HGAGTFTSDLSKQLEEEAVRLFIEFLKNGGPSSGA

SEQ ID NO: 174 - protein, 34 aa, Artificial Sequence
  Description of Artificial Sequence Exendin Agonist
HGEATFTSDLSKQMEEEAVRLFIEWLKNGGPSSG

SEQ ID NO: 175 - protein, 33 aa, Artificial Sequence
  Description of Artificial Sequence Exendin Agonist
HGEGTFTSALSKQMEEEAVRLFIEWLKNGGPSS

SEQ ID NO: 176 - protein, 32 aa, Artificial Sequence
  Description of Artificial Sequence Exendin Agonist
AGEGTFTSDLSKQMEEEAVRLFIEWLKNGGPS

SEQ ID NO: 177 - protein, 32 aa, Artificial Sequence
  Description of Artificial Sequence Exendin Agonist
HGAGTFTSDLSKQLEEEAVRLFIEFLKNGGPS

SEQ ID NO: 178 - protein, 31 aa, Artificial Sequence
  Description of Artificial Sequence Exendin Agonist
HGEATFTSDLSKQMEEEAVRLFIEWLKNGGP

SEQ ID NO: 179 - protein, 30 aa, Artificial Sequence
  Description of Artificial Sequence Exendin Agonist
HGEGTFTSALSKQLEEEAVRLFIEFLKNGG

SEQ ID NO: 180 - protein, 29 aa, Artificial Sequence
  Description of Artificial Sequence Exendin Agonist
AGEGTFTSDLSKQLEEEAVRLFIEFLKNG

SEQ ID NO: 181 - protein, 38 aa, Artificial Sequence
  Description of Artificial Sequence Exendin Agonist
HGAGTFTSDLSKQMEEEAVRLFIEWLKNGGXSSGAXXX

SEQ ID NO: 182 - protein, 38 aa, Artificial Sequence
  Description of Artificial Sequence Exendin Agonist
HGEATFTSDLSKQMEEEAVRLFIEWLKNGGPSSGAXXX

SEQ ID NO: 183 - protein, 37 aa, Artificial Sequence
  Description of Artificial Sequence Exendin Agonist
HGEGTFTSALSKQMEEEAVRLFIEWLKNGGXSSGAXX

SEQ ID NO: 184 - protein, 36 aa, Artificial Sequence
  Description of Artificial Sequence Exendin Agonist
AGEGTFTSDLSKQMEEEAVRLFIEWLKNGGXSSGAX

SEQ ID NO: 185 - protein, 35 aa, Artificial Sequence
  Description of Artificial Sequence Exendin Agonist
HGAGTFTSDLSKQMEEEAVRLFIEWLKNGGPSSGA

SEQ ID NO: 186 - protein, 30 aa, Artificial Sequence
  Description of Artificial Sequence Exendin Agonist
HGDATFTSDLSKQMEEEAVRLFIEWLKNGG

SEQ ID NO: 187 - protein, 39 aa, Artificial Sequence
  Description of Artificial Sequence Exendin Agonist
AGEGTFTSDLSKQMEEEAVRLFIEWLKNGGPSSGAPPPS

SEQ ID NO: 188 - protein, 39 aa, Artificial Sequence
  Description of Artificial Sequence Exendin Agonist
AGAGTFTSDLSKQLEEEAVRLFIEFLKNGGPSSGAPPPS